SEQUENCE LISTING (4 sequences)

SEQ ID NO: 1 - DNA, 20 bp, Artifical Sequence
  Synthetic Polynucleotide
cccaaaagggtcttttcaca

SEQ ID NO: 2 - DNA, 20 bp, Artifical Sequence
  Synthetic Polynucleotide
cacagagccatgaccagaca

SEQ ID NO: 3 - DNA, 25 bp, Artifical Sequence
  Synthetic Polynucleotide
ctcacagtcacatactacctctgac

SEQ ID NO: 4 - DNA, 20 bp, Artifical Sequence
  Synthetic Polynucleotide
atgggatttcatggaagcaa

(19) United States
(12) Patent Application Publication
(10) Pub. No.: US 2019/0276899 A1
(43) Pub. Date: Sep. 12, 2019

(54) DOT1L INHIBITION IN PATIENTS WITH MN1-HIGH AML

(71) Applicant: The Regents of the University of Colorado, A Body Corporate, Denver, CO (US)

(72) Inventors: Kathrin M. BERNT, Aurora, CO (US); Tobias NEFF, Aurora, CO (US)

(21) Appl. No.: 16/402,891

(22) Filed: May 3, 2019

Related U.S. Application Data
(63) Continuation of application No. 15/327,341, filed on Jan. 18, 2017, now abandoned.
(60) Provisional application No. 62/026,583, filed on Jul. 18, 2014.

(51) Int. Cl. C12Q 1/6886; A61P 35/02; A61K 31/7076; A61K 31/7064; G01N 33/574

(52) U.S. Cl. CPC C12Q 1/6886; A61P 35/02; A61K 31/7076; A61K 31/7064; C12Q 2600/156; G01N 2333/4703; G01N 2800/52; C12Q 2600/106; C12Q 2600/158; G01N 33/57426

(57) ABSTRACT

The present disclosure relates to methods of treating AML associated with elevated levels of MN1 and HOXA9 expression by administering one or more DOT1L inhibitors and pharmaceutical compositions to subjects in need thereof.

### RELATED APPLICATIONS

This application is a continuation of U.S. application Ser. No. 15/327,341, filed Jan. 18, 2017, which is a U.S. National Phase Application, filed under 35 U.S.C. § 371, of International Application No. PCT/US2015/040982, filed Jul. 17, 2015, which claims the benefit under 35 U.S.C. 119(e) of U.S. Provisional Application No. 62/026,583, filed Jul. 18, 2014, and entitled “DOT1L Inhibition in Patients with MN1-High AML”, the entire contents of each of which are incorporated herein by reference in their entireties.

### FEDERALLY SPONSORED RESEARCH

This invention was made with government support under NIH-NHLBI grant K08: HL102264-01 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on May 3, 2019, is named “EPIZ-063_CO1US_SeqList.txt” and is 1,167 bytes in size.

### BACKGROUND OF INVENTION

The Meningeoma-1 (MN1) gene is frequently overexpressed in acute myeloid leukemia (AML), and associated with a poor prognosis (Haferlach et al., 2012; Heuser et al., 2006; Langer et al., 2009; Metzeler et al., 2009; Xiang et al., 2013). High MN1 expression occurs across multiple cytogenetic and molecular subgroups of AML, with few consistent associations (Carella et al., 2007; Haferlach et al., 2012; Xiang et al., 2013). Two distinct subtypes of AML are negatively associated with high MN1 expression levels: AML with mutations in nucleophosmin 1 (NPM1c, (Haferlach et al., 2012; Heuser et al., 2006; Langer et al., 2009; Metzeler et al., 2009; Xiang et al., 2013), and AML with a translocation of the mixed lineage leukemia gene, MLL (Carella et al., 2007; Haferlach et al., 2012).

In contrast, the highest expression levels of MN1 have been reported in patients with an inversion of chromosome 16 (inv(16) (Carella et al., 2007; Haferlach et al., 2012)), and 100% of inv(16) AML overexpresses MN1. In apparent contradiction to the poor outcome reported for AML patients with high MN1 expression, inv(16) AML has a favorable prognosis. However, inv16 AML represents only a small subgroup of MN1high AML. A second subgroup associated with higher than average MN1 expression levels is AML with complex karyotype (Haferlach et al., 2012). Despite the relatively good prognosis of inv16 AML, outcomes for MN1high AML as a whole are poor.

### SUMMARY OF THE INVENTION

Aspects of the disclosure relate to methods and compositions for treating AML associated with MN1 overexpression (often associated with poor prognosis). Aspects of the disclosure are based, at least in part, on the determination that AML associated with MN1 overexpression, or MN1 and HOXA9 overexpression is responsive to the inhibition of DOT1L activity. Accordingly, in some embodiments, a subject having AML associated with overexpression of the MN1 gene, or overexpression of MN1 and HOXA9 genes can be treated with one or more DOT1L inhibitor compounds as described herein. In some embodiments, a subject diagnosed with AML and having a genotype that is associated with the overexpression of MN1, or overexpression of MN1 and HOXA9 can be treated with one or more DOT1L inhibitor compounds as described herein. In some embodiments, a subject having one or more deletions of 5q and 7q chromosomal regions can be treated with one or more DOT1L inhibitor compounds as described herein. In some embodiments, a subject having deletions of both 5q and 7q chromosomal regions can be treated with one or more DOT1L inhibitor compounds as described herein. In some embodiments, a subject having one or more deletions within the 5q and/or 7q chromosomal regions can be treated with one or more DOT1L inhibitor compounds as described herein. In some embodiments, a subject having one or more symptoms of AML associated with one or more deletions of 5q and 7q chromosomal regions can be treated with one or more DOT1L inhibitor compounds as described herein. In some embodiments, a subject having one or more symptoms of AML associated with deletions in both 5q and 7q chromosomal regions (e.g., both 5q and 7q are deleted) can be treated with one or more DOT1L inhibitor compounds as described herein.

Accordingly, aspects of the disclosure provide methods and compositions for assisting in the treatment of AML. In some embodiments, aspects of the disclosure are useful to identify AML patients that are responsive to treatment with one or more DOT1L inhibitor compounds. In some embodiments, a subject having one or more clinical symptoms, gene expression markers, and/or karyotypic indicia of AML associated with high MN1, or high MN1 and high HOXA9 expression is identified as a candidate for treatment with a DOT1L inhibitor compound (e.g., as a subject in need of treatment with a DOT1L inhibitor compound). In some embodiments, the subject is treated with one or more DOT1L inhibitor compounds as described herein.

In some embodiments, a subject at risk of developing AML associated with high MN1 expression, or high MN1 and high HOXA9 expression can be treated with one or more DOT1L inhibitor compounds to prevent or slow the progression of the disease.

Non-limiting examples of DOT1L inhibitor compounds include a compound of formula:

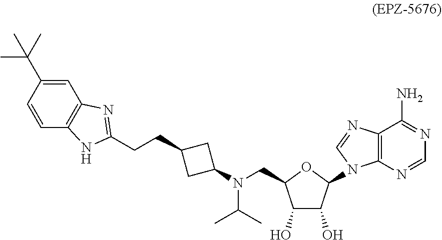 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, prodrug, metabolite, polymorph, solvate, or stereoisomer thereof, and a compound of formula:

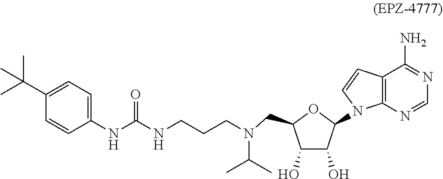 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, prodrug, metabolite, polymorph, solvate, or stereoisomer thereof. However, it should be appreciated that other DOT1L inhibitors as described herein can be used.

Accordingly, the present disclosure provides methods and compositions for treating, preventing, and/or alleviating one or more symptoms of certain AMLs by administering to a subject in need thereof a therapeutically effective amount of a DOT1L inhibitor.

In some embodiments, the leukemia is characterized by one or more deletions within 5q and/or 7q chromosomal regions. In another aspect, a subject having AML has an elevated level of MN1, or an elevated level of MN1 and HOXA9.

In some embodiments, the present disclosure provides a method for treating, preventing, and/or alleviating one or more symptoms of AML in a subject comprising: obtaining a sample from the subject and detecting the level of MN1 and HOXA9 in the sample, wherein an elevated level of MN1, or an elevated level of MN1 and HOXA9 indicates the subject is responsive to a DOT1L inhibitor. In some embodiments, one or more DOT1L inhibitor compounds are administered to the subject in a therapeutically effective amount.

In some embodiments, the present disclosure provides a method for treating, preventing, and/or alleviating one or more symptoms of AML in a subject comprising: obtaining a sample from the subject; detecting the presence of a genetic lesion in 5q and/or 7q in the sample; and administering to the subject a therapeutically effective amount of one or more DOT1L inhibitors when said genetic lesion is present in the sample.

In any of the methods described herein, the sample can be selected from bone marrow, peripheral blood cells, blood, cerebrospinal fluid, skin lesions, chloroma biopsy, plasma, serum, urine, saliva, a cell, or other suitable source.

Accordingly, in some embodiments the present disclosure provides a method for treating a leukemia characterized by deletions in the 5q and/or 7q chromosomal regions by administering to a subject in need thereof a therapeutically effective amount of a DOT1L inhibitor compound.

In another aspect, the disclosure provides methods of selecting a therapy for a subject having leukemia. In some embodiments, a method includes detecting the presence of (a) elevated levels of MN1, or elevated levels of MN1 and HOXA9, and/or (b) one or more deletions in the5q and/or 7q chromosomal regions in a sample from the subject; and selecting, based on the presence of (a) and/or (b) in the sample, a DOT1L inhibitor for treating leukemia. In some embodiments, the method further includes administering to the subject a therapeutically effective amount of the DOT1L inhibitor.

In another aspect, a method of treatment is provided for a subject in need thereof, the method comprising detecting the presence of (a) elevated levels of MN1, or elevated levels of MN1 and HOXA9, and/or (b) one or more deletions in the 5q and/or 7q chromosomal regions in a sample from the subject; and treating the subject based on the presence of (a) and/or (b) with a therapy that includes administering to the subject a therapeutically effective amount of a DOT1L inhibitor.

In some aspects, a therapeutically effective amount of one or more DOT1L inhibitor compounds can be formulated with a pharmaceutically acceptable carrier for administration to a mammal, for example a human subject, for use in treating or preventing leukemia (e.g., AML associated with elevated MN1, or elevated MN1 and HOXA9 and/or 5q and/or 7q deletions).

Accordingly, in certain embodiments, the compounds of the present disclosure are useful for treating, preventing, or reducing the risk of leukemia or for the manufacture of a medicament for treating, preventing, or reducing the risk of leukemia. In some embodiments, compounds or formulations described herein can be administered, for example, via oral, parenteral, otic, ophthalmic, nasal, or topical routes, to provide an effective amount of the compound to the mammal.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing aspects of the present disclosure, suitable methods and materials are described herein. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE DRAWINGS

FIGS. 1A-1D illustrate that the MN1 cooperating program (including HoxA9) is dependent on functional Dotll using a murine genetic loss of function model. FIG. 1A shows total white blood cell count (WBC), hemoglobin (Hb) and platelet count (Plt) in Dot1lf/f (f/f, 10 mice) and MxCreDot1lf/f (−/−, 9 mice) mice 3 week after the induction of Cre. *p<0.05. FIG. 1B shows an expression array of sorted lineage⁻ Sca-1⁺ cKit⁺ (LSK cells, enriched for hematopoietic stem and uncommitted progenitors) from Dot1lf/f (ff) and MxCreDot1lf/f (−/−) 12 days after mouse induction of Cre. Shown are all probe sets / genes with differential expression at p=0.01 (393 genes), as well as a list of the top 30 differentially expressed probe sets and Meis1. HoxA9 is among the top 30 down-regulated genes after loss of functional Dot1l in normal LSK cells. N=6 mice per group. FIG. 1C shows a Venn diagram of genes associated with H3K79 dimethylation in LSK cells by ChIP-Seq (Bernt et al.) and genes down-regulated after loss of Dot1l in LSK cells. FIG. 1D shows a gene set enrichment analysis (GSEA) showing enrichment of the MN1 cooperating signature in Dot1lf/f versus Dot1l−/− LSK cells.

FIGS. 2A-2F show that loss of Dot1L leads to decreased growth, increased differentiation, and apoptosis of MN1 driven, common myeloid progenitor (CMP) derived murine leukemia cells. FIG. 2A shows serial replating of MN1 transformed CMPs (^{MN1}CMP-T) after Cre-induced loss of Dot1l. Left plot: number of colonies per 500 plated cells, right plot: total cell number. N=3 independent experiments, error bars: SEM. FIG. 2B shows serial replating of MN1 transformed CMPs (^{MN1}CMP-L) after Cre-induced loss of Dot1l. Left plot: number of colonies per 500 plated cells, right plot: total cell number. Dot1l^{ff}: bulk population from 3 independent experiments, Dot1l^{-/-}: 2 bulk population and 4 individually picked clones (due to outgrowth of non-deleted cells) from 3 independent experiments, error bars: SEM. FIG. 2C shows methylcellulose colony and leukemia cell morphology (Wright Giemsa Stain) of MN1 transformed CMPs 14 days after transduction with Cre. FIG. 2D shows CD11b expression in MN1 transformed CMPs 3 weeks after deletion of Dot1l. Dot1l^{ff}: bulk population from 3 independent experiments, Dot1l^{-/-}: 2 bulk population and 4 individually picked clones (due to outgrowth of non-deleted cells) from 3 independent experiments, error bars: SEM. FIG. 2E shows apoptosis (Annexin staining) in MN1 transformed CMPs 3 weeks after deletion of Dot1l. Dot1l^{ff}: bulk population from 3 independent experiments, Dot1l^{-/-}: 2 bulk population and 4 individually picked clones (due to outgrowth of non-deleted cells) from 3 independent experiments, error bars: SEM. FIG. 2F shows cell cycle distribution (EdU incorporation/DAPI staining) in MN1 transformed CMPs 3 weeks after deletion of Dot1l. Dot1lff: bulk population from 3 independent experiments, Dot1l^{-/-}: 2 bulk population and 4 individually picked clones (due to outgrowth of non-deleted cells) from 3 independent experiments, error bars: SEM.

FIGS. 3A-3D illustrate that CMP derived murine MN1 driven leukemia is dependent on functional DOT1L in vivo. FIG. 3A shows leukemic burden (% of GFP positive cells in the peripheral blood) in recipients on day 20 after injection of MNl in vitro transformed CMPs (^{MN1}CMP-T) transduced with Cre (Dot1l^{-/-}) or control (Dot1l^{f/f}) vector. N=5 (Dot1l^{-/-}) to 6 (Dot1l^{f/f}) mice from 2 individual experiments, error bars: SEM. FIG. 3B shows survival of recipients of MN1 in vitro transformed CMPs (^{MN1}CMP-T) transduced with Cre (Dot1l^{-/-}) or control (Dot1l^{f/f}) vector. N=6 (Dot1l^{-/-}) to 7 (Dot1l^{f/f}) mice from 2 individual experiments. FIG. 3C shows leukemic burden (% of GFP positive cells in the peripheral blood) in recipients on day 20 after injection of MN1 driven, CMP derived leukemias (^{MN1}CMP-L) transduced with Cre (Dot1l^{-/-}) or control (Dot1l^{f/f}) vector. N=9 mice per group from 2 individual experiments, error bars: SEM. FIG. 3D shows survival of recipients of MN1 driven, CMP derived leukemias (^{MN1}CMP-L) transduced with Cre (Dot1l^{-/-}) or control (Dot1l^{f/f}) vector. N=9 (Dot1l^{-/-}) to 11 (Dot1l^{f/f}) mice from 2 individual experiments.

FIGS. 4A-4C show that the MN1 cooperating program is down-regulated after loss of Dot1l in MN1 transformed CMPs (^{MN1}CMP-T). FIG. 4A shows qPCR for HoxA9 and Meis1 in ^{MN1}CMP-T 7 days after transduction with Cre. N=3 independent experiments, error bars: SEM. FIG. 4B shows RNA-sequencing of sorted ^{MN1}CMP-T 7 days after transduction with Cre. Shown are all probe sets/genes with differential expression at p=0.01, as well as a list of the top 30 differentially expressed probe sets and Meis1. N=6 mice per group. FIG. 4C shows GSEA showing enrichment of the MN1 cooperating signature defined by Heuser et al in Dot1lf/f versus Dot1l−/− ^{MN1}CMP-T.

FIGS. 5A-5C show that hematopoietic stem cells are inferior cells of origin for MN1, but not MLL-AF9 induced leukemias. FIG. 5A shows survival of primary recipients of MN1 in vitro transformed CMPs (^{MN1}CMP-T, including limiting dilution), LSK-SLAM (^{MN1}SLAM-T) and LT-HSCs (^{MN1}LTHSC-T). ^{MN1}CMP-T 100k: n=13, 3 individual experiments; ^{MN1}CMP-T 10k: n=3; ^{MN1}CMP-T 1k: n=3; ^{MN1}SLAM-T: n=8, 2 individual experiments; ^{MN1}LTHSC-T: n=7, 2 individual experiments. ^{MN1}CMP-T 100k versus ^{MN1}CMP-T and ^{MN1}SLAM-T: p<0.0001 (Mantel-Cox). FIG. 5B shows survival of primary recipients of MLL-AF9 (MA9) in vitro transformed CMPs (^{MA9}CMP-T, n=6), and LSK-SLAM (^{MA9}SLAM-T, n=5). p=not significant (Mantel-Cox). FIG. 5C shows survival of secondary recipients MN1 driven leukemias based on cell of origin: ^{MN1}CMP-L, n=10), LSK-SLAM (^{MN1}SLAM-L, n=11), and LT-HSCs (^{MN1}LTHSC-L). p=not significant (Mantel-Cox)

FIGS. 6A-6J show that ^{MN1}HSC-T grow independently of Dot1l in vitro but not in vivo. FIG. 6A shows serial cell counts of Dot1l^{f/f} and Dot1l^{-/-MN1}HSC-T. n=5 individual experiments (3LT-HSC, 2 SLAM). There were no statistically significant differences between Dot1l^{f/f} and Dot1l^{-/-MN1}HSC-T (or LT-SHC and SLAM derived ^{MN1}HSC-T, data not shown). FIG. 6B shows methylcellulose colony morphology of MN1 or MLL-AF9 transformed LT-HSCs 9 days after transduction with Cre. FIG. 6C shows genomic PCR for floxed (flox) and deleted (del) Dot1l alleles in MN1 or MLL-AF9 transformed LT-HSCs 14 days after transduction with Cre. FIG. 6D shows qPCR for Dot1l, HoxA9 and Meis1 in ^{MN1}HSC-T, error bars: SEM, each bar represents fold-change in Dot1l^{-/-} compared to Dot1l^{f/f} (set to 1), error bars: SEM, *p<0.01, ns: p=not significant (2-sided t-test Dot1l^{-/-} vs Dot1l^{f/f}). FIG. 6E shows qPCR for HoxA9 in ^{MN1}CMP-T and ^{MN1}HSC-T, each bar represents fold-change in the indicated population compared to ^{MN1}HSC-T control (set to 1, first bar), error bars: SEM, *p<0.01, ns: p=not significant (2-sided t-test of indicated population vs ^{MN1}HSC-T control). FIG. 6F shows leukemic burden in primary recipients (measured as % GFP+ cell in the peripheral blood) on day 38 after transplantation with ^{MN1}HSC-T and ^{MLL-AF9}HSC-T transduced with either Cre or Control (Co). ^{MN1}HSC-T: n=6 per group, 2 independent experiments; ^{MLL -AF9}HSC-T: n=5(Cre) and 4(Co) per group, error bars: SEM, *p<0.001 (2-sided t-test). FIG. 6G shows survival of primary recipients of MN1 in vitro transformed ^{MN1}HSC-T transduced with either Cre or control, n=6 per group, 2 independent experiments, p<0.05 (Mantel-Cox, when compared to all ^{MN1}HSC-T historic controls not significant, refer to FIG. 13F). *: failure to rearrange both Dot1l floxed alleles confirmed by genomic PCR FIG. 6H shows qPCR for Dot1l, HoxA9 and Meis1 in

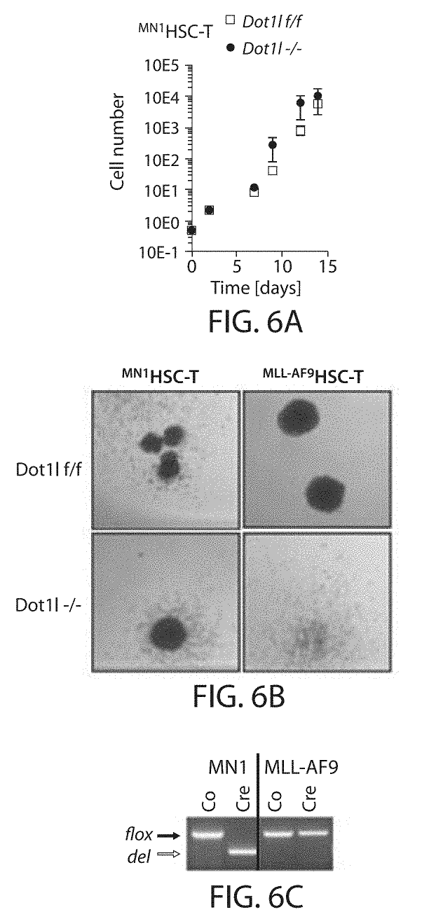

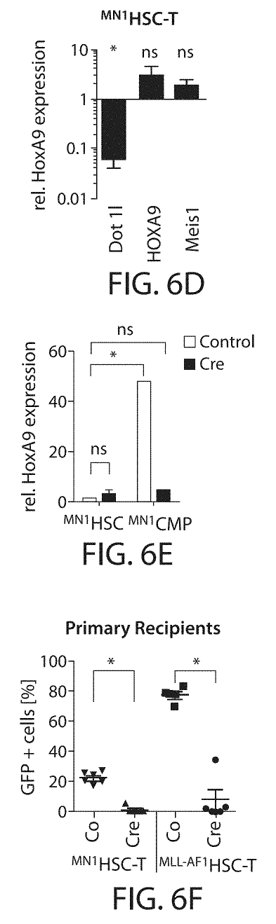

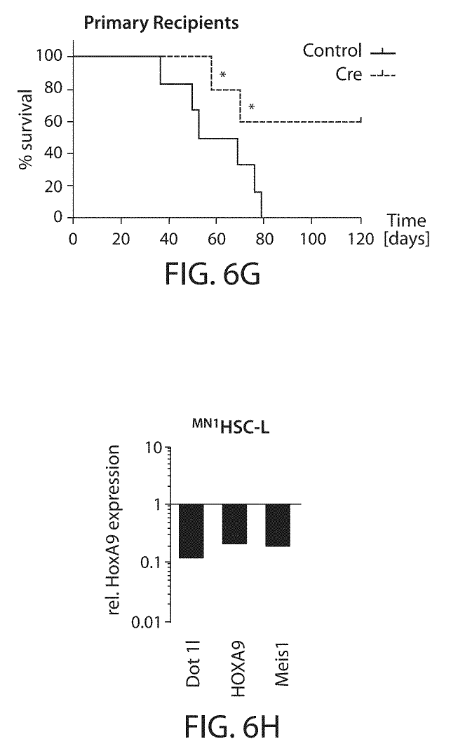

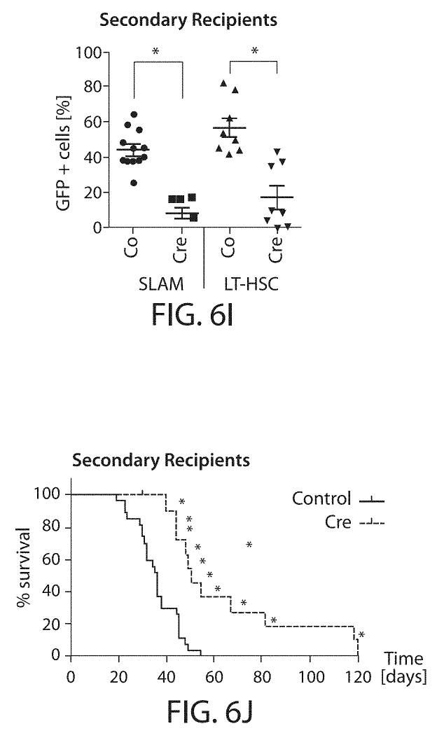

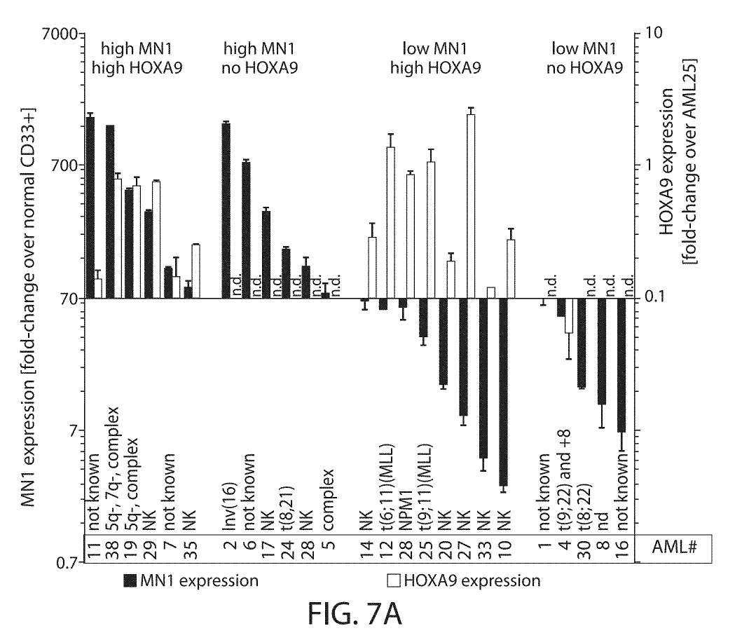

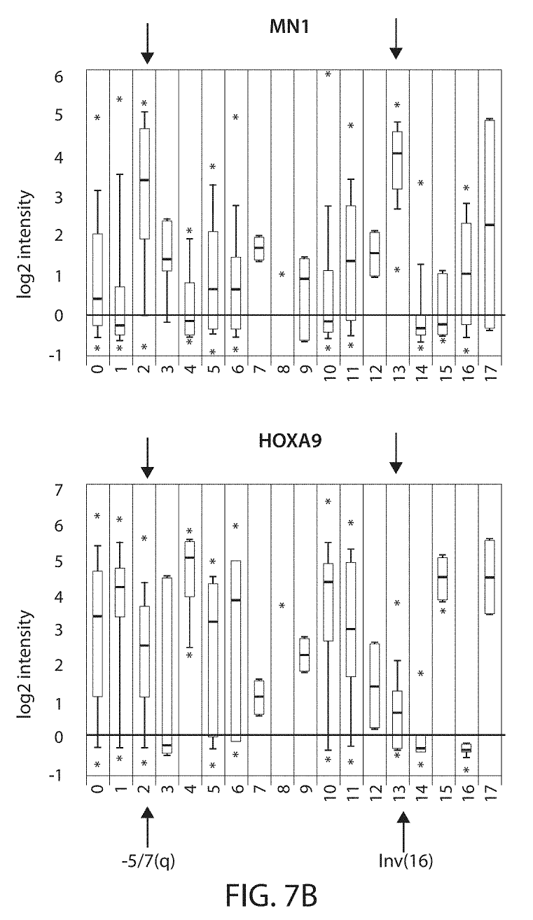

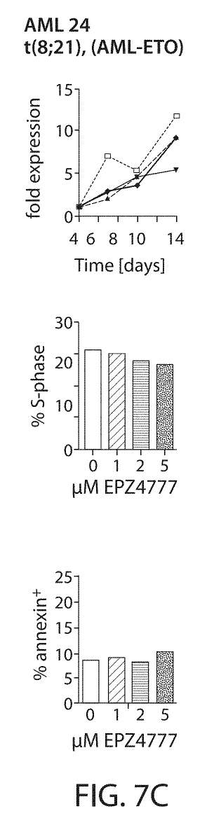

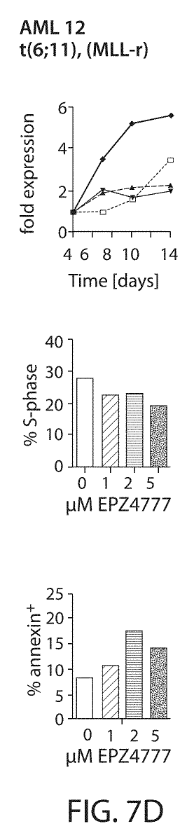

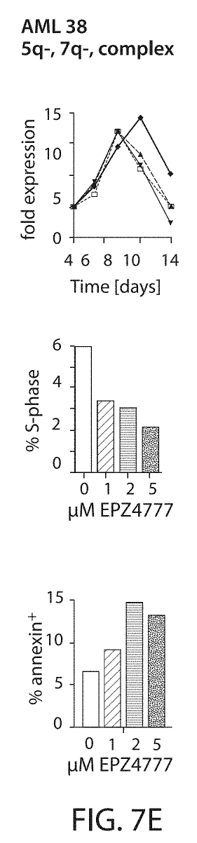

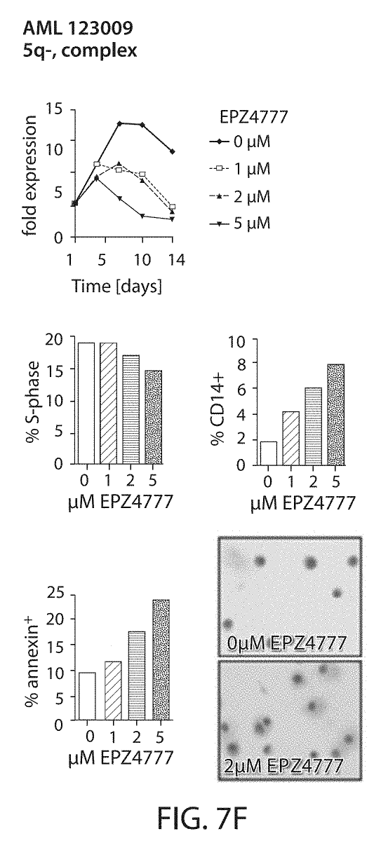

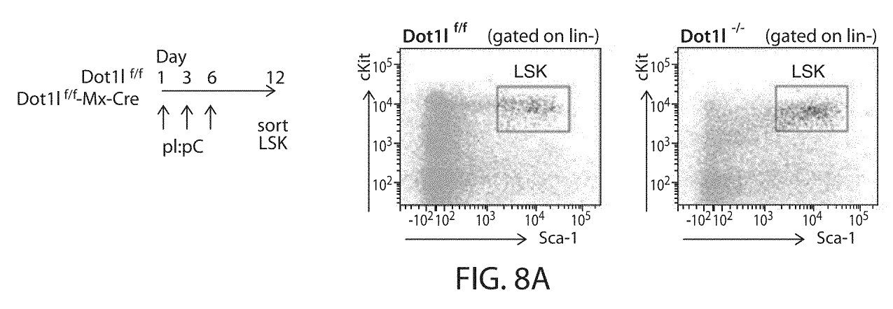

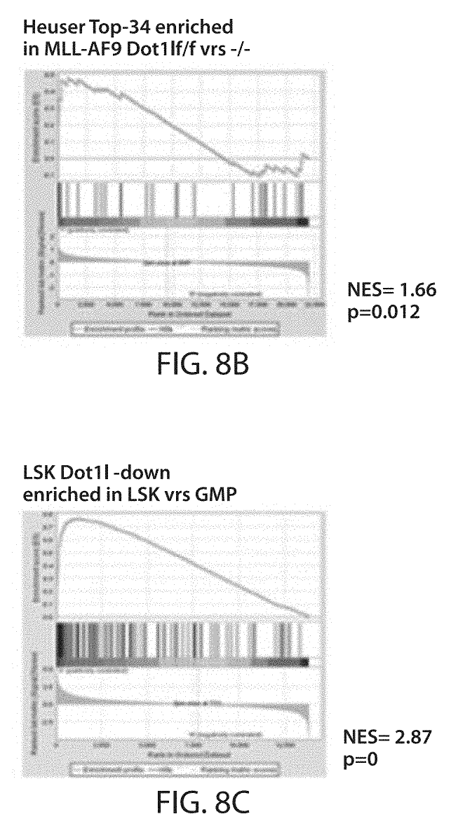

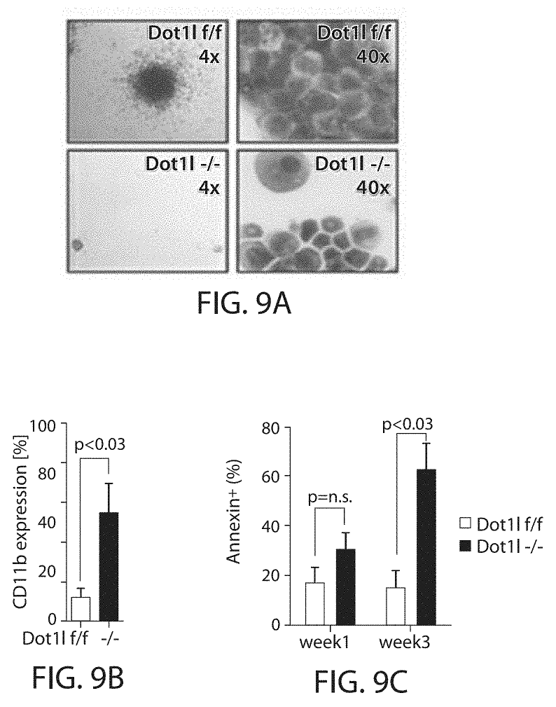

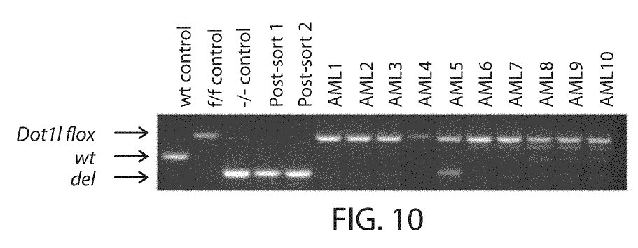

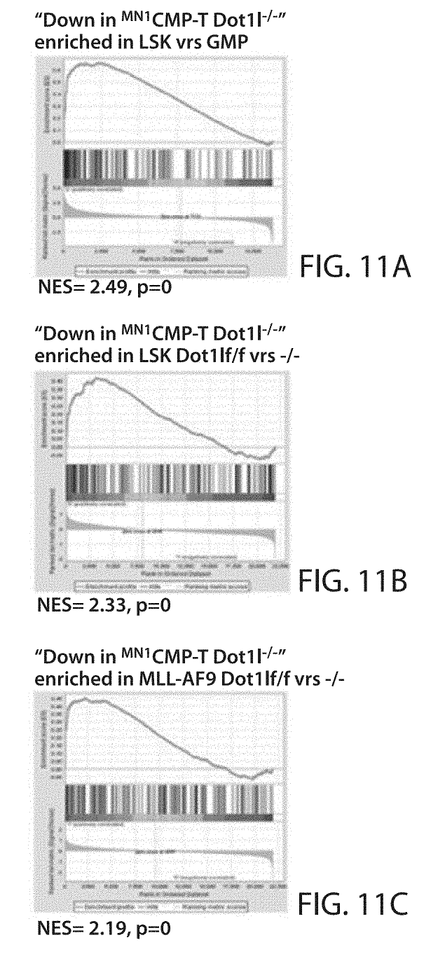

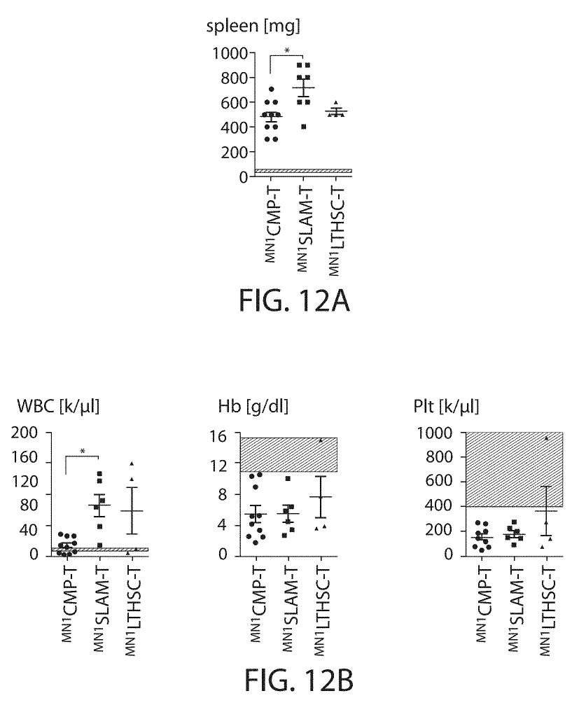

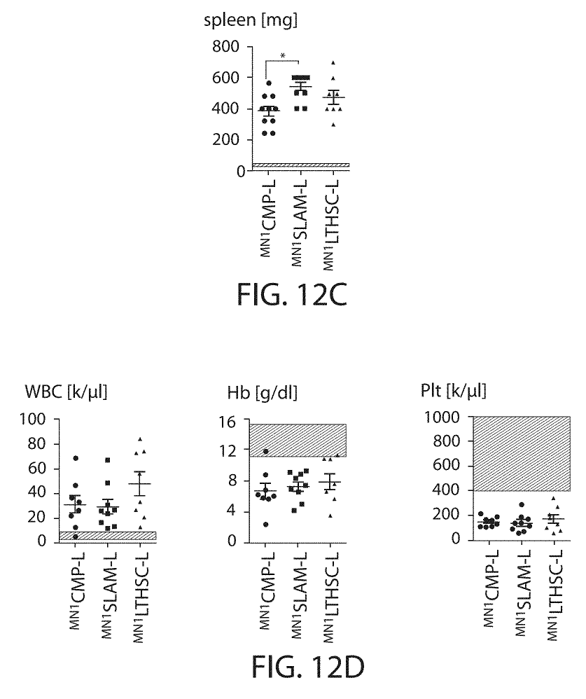

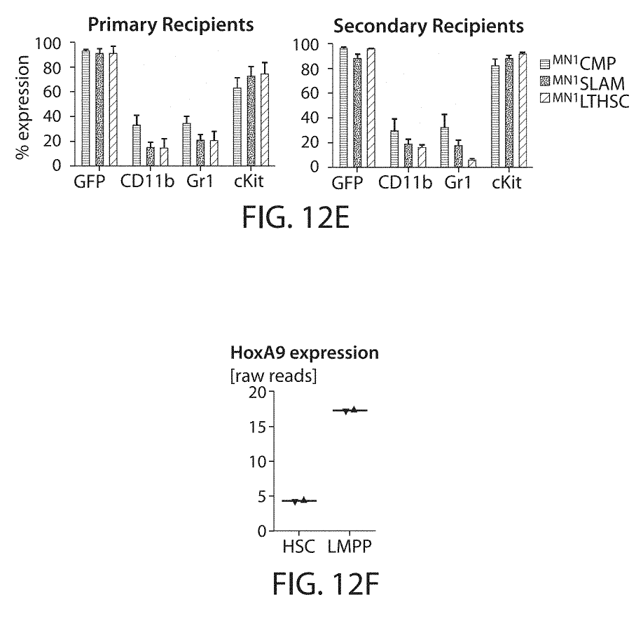

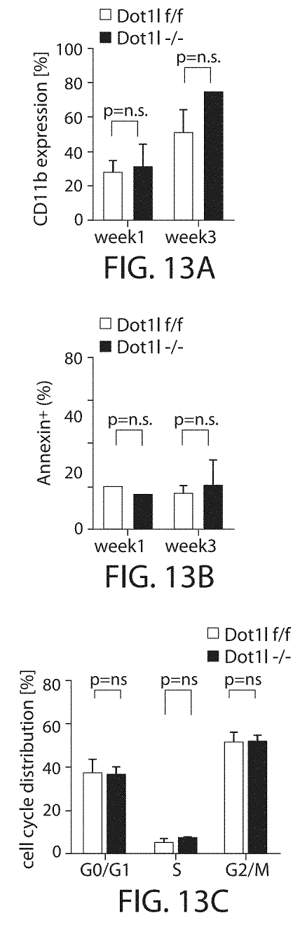

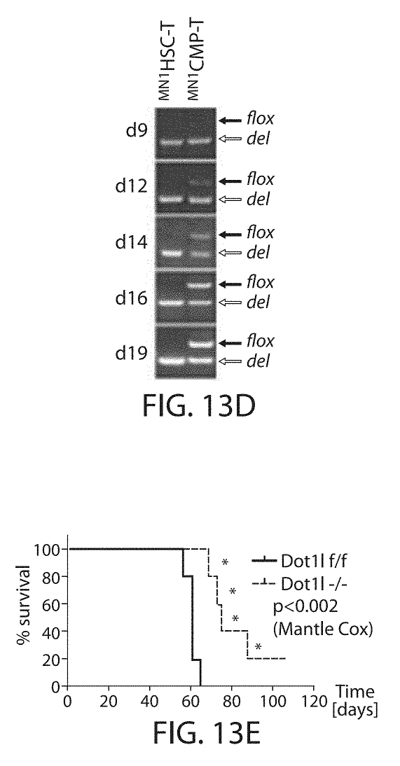

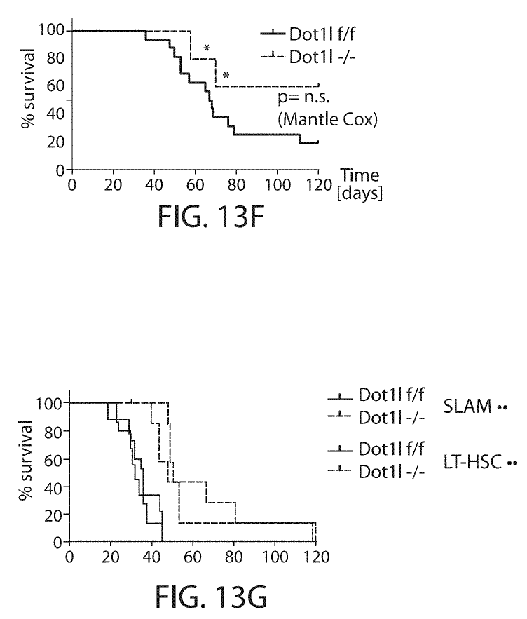

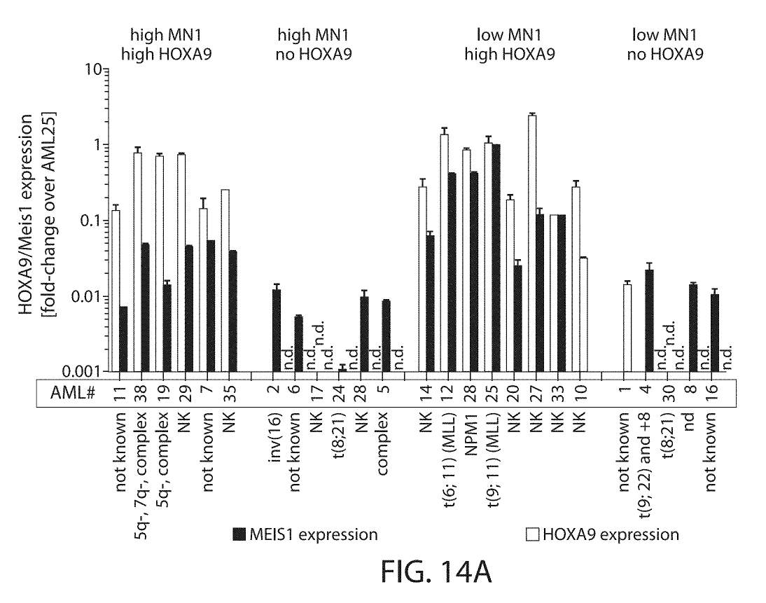

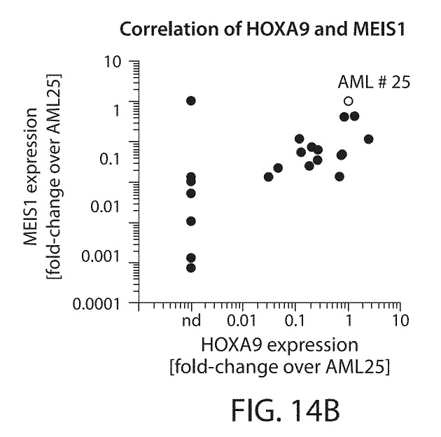

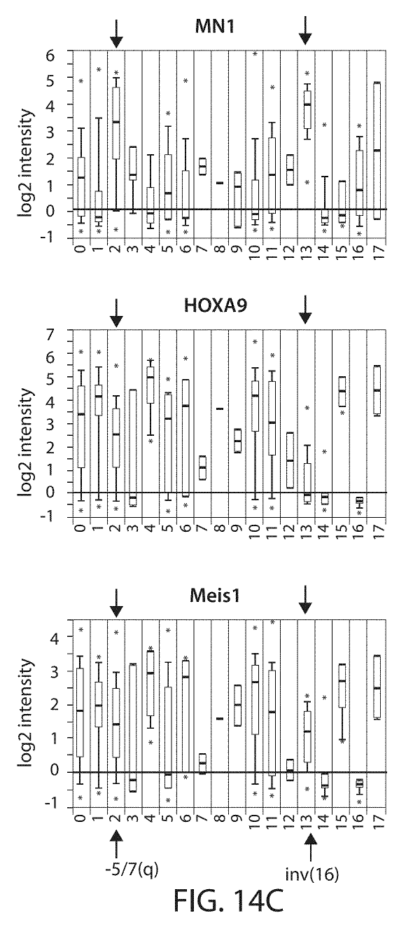

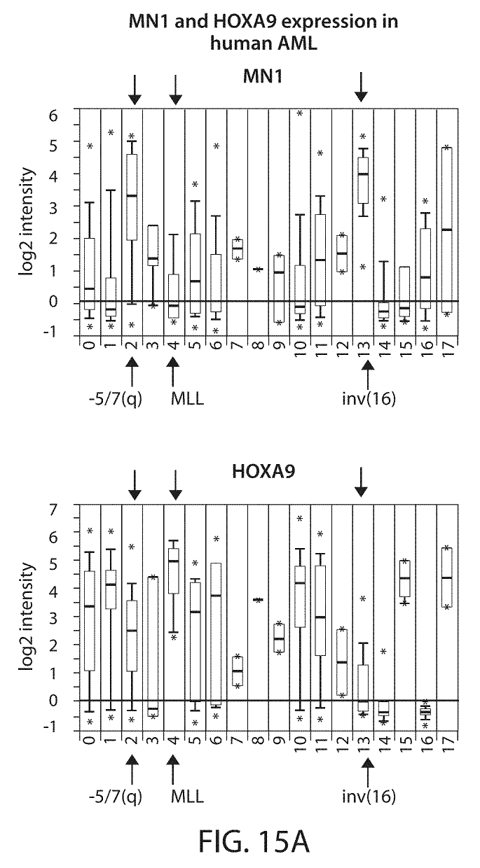

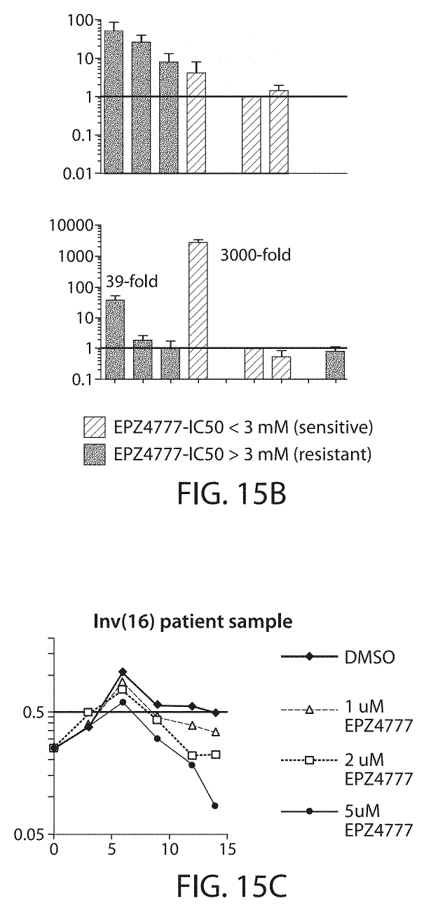

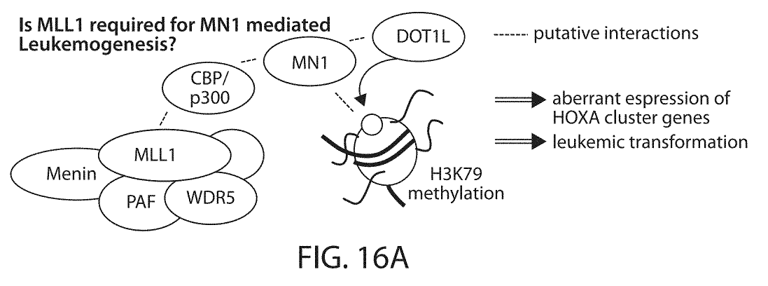

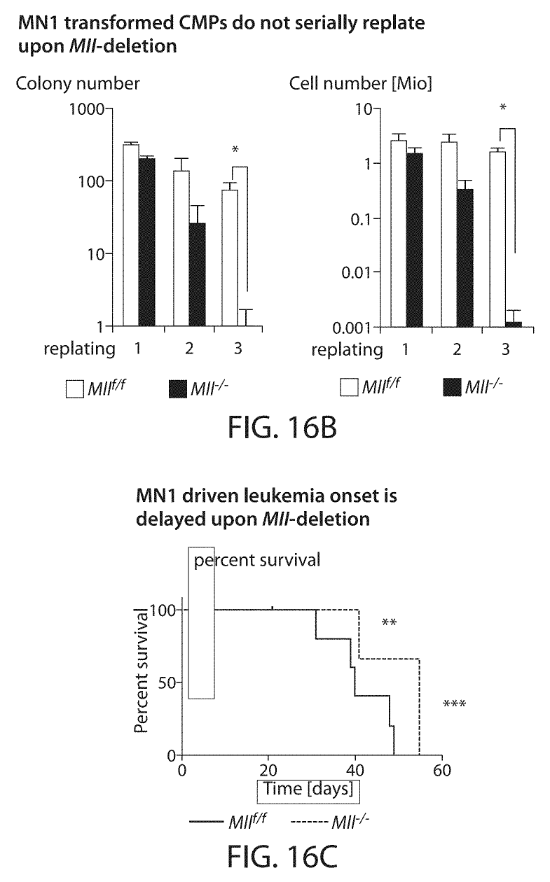

^{MN1}HSC-L (needs repeat). FIG. 61 shows leukemic burden in secondary recipients (measured as % GFP+ cell in the peripheral blood) on day 38 after transplantation with ^{MN1}HSC-L (either LT-HSC or LKS-SLAM derived) transduced with either Cre or Control (Co). n=12 (LSK-SLAM-Control), 7 (LSK-SLAM-Cre), 8 (LT-HSC-Control) and 8 (LT-HSC-Cre) from 3 (LT-HSC) and 2 (LSK-SLAM) primary leukemias, 6 independent experiments; error bars: SEM, *p<0.001 (2-sided t-test). FIG. 6J shows survival of secondary recipients of ^{MN1}HSC-L transduced with either Cre or control, n=12 (LSK-SLAM-Control), 7 (LSK-SLAM-Cre), 8 (LT-HSC-Control) and 8 (LT-HSC-Cre) from 3 (LT-HSC) and 2 (LSK-SLAM) primary leukemias, 6 independent experiments, p<0.0001; *:failure to rearrange both Dot1l floxed alleles confirmed by genomic PCR.

FIGS. 7A-7F show that a subgroup of MN1high AML patient samples expresses HOXA9 and is sensitive to DOT1L inhibition. FIG. 7A shows qPCR analysis of MN1 and HOXA9 in 24 initial diagnostic AML samples (>80% CD33⁺). MN1 expression is shown dichotomized at the median, values refer to fold enrichment compared to normal CD33⁺ myeloid progenitors. HOXA9 values are plotted as fold-enrichment compared to AML25 (MLL-rearranged, with known high HOXA9 expression). Error bars: SEM of 3 technical replicates (still need to insert). n.d.: not detected. FIG. 7B shows MN1 and HOXA9 expression by genotype in Wouters Leukemia data set (Oncomine™). Full legend: 0: Not determined (90), 1: +8 (20); 2: −5/7(q) (29); 3: −9q (6); 4: 11q23 (10); 5: Complex (13); 6: Failure (12); 7: MDS −7(q) (2); 8: MDS −Y (1); 9: MDS Complex (3); 10: Normal (187); 11: Other (53); 12: abn(3q) (2); 13: idt(16) (34); 14: t(15;17) (21); 15: t(6;9) (6); 16: t(8;21) (35); 17: t(9;22) (2). N=526 AML samples. FIGS. 7C-7F) show exposure of 4 primary patients AML samples to the DOT1L inhibitor EPZ4777 at the indicated concentrations. FIG. 7C: AML24 (AML/ETO, negative control), FIG. 7D: AML12 (MLL-rearranged, positive control), FIG. 7E: AML28 (high MN1/HOXA9, complex karyotype with 5q-/7q-), FIG. 7F: AML123009 (high MN1/HOXA9, complex karyotype with 5q-). Shown are fold expansion over a 14 day culture period (serial cell counts and Trypan Blue staining, top panel, error bars=duplicate counts), cell cycle (% cells in S-phase, EdU incorporation, middle panel), apoptosis (Annexin staining, lower panel) and differentiation (CD14 expression by flow cytometry, Write Giemsa stain on cytospin, FIG. 7F).

FIGS. 8A-8C illustrate an experimental design scheme and representative flow sort to determine Dot1l dependent gene set in LSK cells. FIG. 8A shows Dot1l^{f/f} (control) and Dot1l^{f/f} Mx-Cre mice, 6 mice per group, were injected with 3 doses of pI:pC on days 1,3 and 6. Mice were sacrificed on day 12 (6 days after the last injection of pI:pC). LSK cells were sorted for gene expression profiling. Flow plots for control mice showed the expected pattern with no or minimal residual effects from pI:pC. Dot1l−/− mice show a beginning decrease in cKit expression particularly in the progenitor compartment, but the LSK cells are still clearly identifiable. FIG. 8B shows GSEA showing enrichment of gene dependent on Dotil in MLL-AF9 driven leukemia (MLL-AF9 Dot1l-down) in Dot1l−/− LSK cells. FIG. 8C shows GSEA showing enrichment of gene down-regulated at the LSK to GMP transition (GMP-down) in Dot1−/− LSK cells.

FIGS. 9A-9C illustrate differentiation and apoptosis in ^{MN1}CMP-T. FIG. 9A shows methylcellulose colony and cell morphology (Wright Giemsa staining) of MN1 transformed CMPs (^{MN1}CMP-T) 27 days after transduction with Cre. FIG. 9B shows CD11b expression in ^{MN1}CMP-T 3 weeks after deletion of Dot1l. n=3 independent experiments. FIG. 9C shows apoptosis (Annexin staining) in ^{MN1}CMP-T 3 weeks after deletion of Dot1l. n=3 independent experiments. Error bars: SEM

FIG. 10 shows the outgrowth of leukemia cells with at least one floxed allele in primary and secondary MN1 driven leukemias (PCR).

FIGS. 11A-11C show gene set enrichment analysis (GSEA) of gene dependent on Dot1l in ^{MN1}CMP-T (“Down in ^{MN1}CMP-T Dot1l^{-/-}”). FIG. 11A shows GSEA showing enrichment of gene dependent on Dotll in ^{MN1}CMP-T in genes down-regulated at the LSK to GMP transition. FIG. 11B shows GSEA showing enrichment of gene dependent on Dot1l in ^{MN1}CMP-T Dot1l−/− versus f/f normal LSK cells. FIG. 11C shows GSEA showing enrichment of gene dependent on Dot1l in ^{MN1}CMP-T in MLL-AF9 Dot1l−/− versus f/f leukemias.

FIGS. 12A-12F provide a detailed analysis of CMP and HSC derived primary and secondary leukemias. FIG. 12A shows spleen weight of primary recipient mice injected with 100,000 ^{MN1}CMP-T, ^{MN1}SLAM-T, or ^{MN1}LT-HSC-T at the time of death. n=11(^{MN1}CMP-T), 6 (^{MN1}SLAM-T), and 3 (^{MN1}LT-HSC-T). FIG. 12B shows complete blood count of primary recipient mice injected with 100,000 ^{MN1}CMP-T, ^{MN1}SLAM-T, or ^{MN1}LT-HSC-T at the time of death. n=11(^{MN1}CMP-T), 6 (^{MN1}SLAM-T), and 3 (^{MN1}LT-HSC-T). FIG. 12C shows spleen weight of secondary recipient mice injected with 100,000 ^{MN1}CMP-L, ^{MN1}SLAM-L, or ^{MN1}LT-HSC-L at the time of death. n=10(^{MN1}CMP-L), 11 (^{MN1}SLAM-L), and 8 (^{MN1}LT-HSC-L). FIG. 12D shows complete blood count of secondary recipient mice injected with 100,000 ^{MN1}CMP-L, ^{MN1}SLAM-L, or ^{MN1}LT-HSC-L at the time of death. n=10(^{MN1}CMP-L), 11 (^{MN1}SLAM-L), and 8 (^{MN1}LT-HSC-L). FIG. 12E shows flow cytometric analysis of the bone marrow of mice from A-D at the time of death. Leukemic burden is estimated by the amount of GFP+ cells in the bone marrow. FIG. 12F shows a graphic representation of HoxA9 RNA-Seq raw reads in HSCs and LMPPs from NCBI GEO accession number GSE50896 (Boiers et al., 2013). Shaded area: normal range. Error bars: SEM. *p<0.05 (ANOVA)

FIGS. 13A-13G show that ^{MN1}HSC-T grow independently of Dot1l in vitro but not in vivo. FIG. 13A shows CD11b expression in MN1 transformed HSCs 1 and 3 weeks after deletion of Dot1l. Bulk population from 3 independent experiments, error bars: SEM. There are no statistically significant differences between Dot1l^{f/f} and Dot1l^{-/- MN1}HSC-T. Interestingly, CD11b expression increases over time in these cultures, a phenomenon we have not seen to this extent in CMP derived cultures. The significance of this finding is unclear, but could relate to the inferior ability of these cells to cause in vivo leukemias. FIG. 13B shows apoptosis (Annexin staining) in MN1 transformed HSCs 1 and 3 weeks after deletion of Dot1l. Bulk population from 3 independent experiments. Error bars: SEM. p=not significant. FIG. 13C shows cell cycle distribution (EdU incorporation/DAPI staining) in MN1 transformed HSCs 1 and 3 weeks after deletion of Dot1l. Bulk population from 3 independent experiments. Error bars: SEM. p=not significant. FIG. 13D shows serial genomic PCR for floxed (flox) and deleted (del) Dot1l alleles in MN1 transformed HSCs (^{MN1}HSC-T) or CMPs (^{MN1}HSC-T) up to 19 days after transduction with Cre. FIG. 13E shows survival of primary recipients of MLL-AF9 in vitro transformed ^{MLL-AF9}HSC-T transduced with either Cre or control, n=5 (cre) and 4 (control) per group, p<0.02*: failure to rearrange both Dot1l floxed alleles confirmed by genomic PCR. FIG. 13F shows survival of primary recipients of MN1 in vitro transformed ^{MN1}HSC-T transduced with either Cre or control, Cre: n=6 per group, 2 independent experiments, Control: n=24, summary of control mice in this experiment and historic controls, p=not significant (Mantel-Cox). *: failure to rearrange both Dot1l floxed alleles confirmed by genomic PCR. FIG. 13G shows survival of secondary recipients of ^{MN1}HSC-L transduced with either Cre or control, n=12 (LSK-SLAM-Control), 7 (LSK-SLAM-Cre), 8 (LT-HSC-Control) and 8 (LT-HSC-Cre) from 3 (LT-HSC) and 2 (LSK-SLAM) primary leukemias, 6 independent experiments, **p<0.001

FIGS. 14A-14C illustrate a subgroup of MN1high AML patient samples that express HOXA9 and are sensitive to DOT1L inhibition. FIG. 14A shows qPCR analysis of HOXA9 and MEIS1 in 25 initial diagnostic AML samples (>80% CD33⁺). HOXA9/MEIS1 expression is plotted as fold-enrichment compared to AML25 (MLL-rearranged, with known high HOXA9/MEIS1 expression). Error bars: SEM of 3 technical replicates. n.d.:not detected. FIG. 14B shows correlation of HOXA9 and MEIS1 expression in 25 initial diagnostic AML samples. FIG. 14C shows MN1, HOXA9 and MEIS1 expression by genotype in Wouters Leukemia data set (Oncomine™). Full legend: 0: Not determined (90), 1: +8 (20); 2: −5/7(q) (29); 3: −9q (6); 4: 11q23 (10); 5: Complex (13); 6: Failure (12); 7: MDS −7(q) (2); 8: MDS −Y (1); 9: MDS Complex (3); 10: Normal (187); 11: Other (53); 12: abn(3q) (2); 13: idt(16) (34); 14: t(15;17) (21); 15: t(6;9) (6); 16: t(8;21) (35); 17: t(9;22) (2). N=526 AML samples.

FIGS. 15A-15C illustrate MN1 correlation with DOT1L dependence. FIG. 15A shows MN1 and HOXA9 expression in human AML and correlation with cytogenetics. Wouters leukemia data set (Oncomine). 0: Not determined (90), 1: +8 (20); 2: −5/7(q) (29); 3: −9q (6); 4: 11q23 (10); 5: Complex (13); 6: Failure (12); 7: MDS −7(q) (2); 8: MDS −Y (1); 9: MDS Complex (3); 10: Normal (187); 11: Other (53); 12: abn(3q) (2); 13: idt(16) (34); 14: t(15;17) (21); 15: t(6;9) (6); 16: t(8;21) (35); 17: t(9;22) (2). N=526 AML samples. FIG. 15B shows HOXA9 expression alone does not predict response to DOT1L inhibitor. Top: HOXA9 expression; Bottom: MN1 expression by qPCR (relative to MV4;11). FIG. 16C shows the response of an inv(16) patient sample to DOT1L inhibitor EPZ4777.

FIGS. 16A-16C show the role of MLL1 in MN1 mediated leukemogenesis. FIG. 16A illustrates a non-limiting model showing Mll-1 is involved in MN1 mediated leukemogenesis. FIG. 16B shoes serial replating of MN1 transformed cells after Cre-mediated deletion of Mll. Colony numbers and cell numbers per 500 plated cells, *p<0.05. FIG. 16C shows survival of recipients of 100 000 MN1 transformed CMPs transduced with Cre (Mll−/−) or control (Mllf/f) vector. *: failure to rearrange both Mll alleles in resultant leukemia.

### DETAILED DESCRIPTION OF THE INVENTION

Aspects of the present disclosure are based in part upon the surprising discovery that DOT1L inhibitors can effectively treat acute myeloid leukemia (AML) associated with overexpression of meningeomal (MN1) and HOXA9 genes (hereinafter referred to as MN1^{high}/HOXA9^{high} AML). In some embodiments, leukemia cells having elevated mRNA or protein levels of MN1 and HOXA9 are sensitive to the DOT1L inhibitors as described herein. Accordingly, the present disclosure provides methods of treating, preventing, or alleviating one or more symptoms of leukemia associated with high MN1 and high HOXA9 in a subject by administering a therapeutically effective amount of a DOT1L inhibitor to the subject. In some embodiments, the present disclosure provides methods of treating, preventing, or alleviating one or more symptoms of leukemia in a subject having one or more genetic lesions associated with high MN1 and high HOXA9 by administering a therapeutically effective amount of a DOT1L inhibitor to the subject. In some embodiments, the present disclosure provides methods of treating, preventing, or alleviating one or more symptoms of AML associated with 5q and/or 7q chromosomal deletions in a subject by administering a therapeutically effective amount of a DOT1L inhibitor to the subject.

Aspects of the disclosure are particularly useful for treating certain forms of AML that have a poor prognosis. Meningeoma-1 (MN1) overexpression in AML typically predicts a poor prognosis. According to aspects of the disclosure MN1 overexpression induces an aggressive myeloid leukemia. In some embodiments, this leukemia is dependent on the expression of a defined gene expression program, including the key components HOXA9 and MEIS1, in a progenitor cell of origin. According to aspects of the disclosure, this gene expression program is controlled by the histone methyltransferase DOT1L. Accordingly, one or more DOT1L inhibitors can be used to inhibit this gene expression program and inhibit one or more steps of disease progression in AML associated with high MN1, or high MN1 and high HOXA9 (and optionally high MEIS1).

In some embodiments, methods and compositions described by this document can be used to treat AML associated with high MN1 but not high HOXA9 (e.g., normal HOXA9, for example, represented by the average or median HOXA9 expression level in a population of patients that do not have cancer, or in one or more non-cancerous cell lines or biological samples, or other reference level indicative of normal HOXA9 expression). For example, in some embodiments a subject having AML characterized by overexpression of MN1 but not overexpression of HOXA9 (e.g., high MN1, normal HOXA9) is responsive to treatment with a DOT1L inhibitor. In some embodiments, high MN1 but not high HOXA9 is detected in a biological sample obtained from a subject and the subject is then identified as a candidate for treatment (e.g., the subject is identified as being responsive to treatment) with a DOT1L inhibitor.

The terms “high MN1” and “high HOXA9” refer to the expression level (e.g., overexpression) of each gene (e.g., MN1 or HOXA9) in a sample (e.g., a biological sample). A biological sample can have high MN1, high HOXA9, or high MN1 and high HOXA9. Overexpression of a gene is generally understood to be elevated expression of a gene (e.g., MN1, HOXA9) relative to a normal expression (e.g., in a normal subject or in a normal reference cell). Overexpression may also refer to increased expression of a gene in one tissue or cell type of a subject relative to a different tissue or cell type within the subject. For example, cancerous bone marrow of a subject having a leukemia (e.g., AML) may have high MN1 (and/or high HOXA9), whereas normal bone marrow from the subject having a leukemia (e.g., AML) may exhibit a normal MN1 (and/or HOXA9) expression level. In some embodiments, overexpression of genes (e.g., high MN1, or high MN1 and high HOXA9) is associated with a disease (e.g., AML, for example AML that is responsive to treatment with a DOT1L inhibitor). It should be appreciated that embodiments described herein in the context of high MN1 and high HOXA9 also can be practiced in the context of high MN1 without high HOXA9.

In some embodiments, the expression level of MN1 in a biological sample having “high MN1” is between about 2-fold and about 5,000-fold higher than a biological sample not having high MN1. In some embodiments, the expression level of MN1 in a biological sample having “high MN1” is between about 10-fold and about 1,000-fold higher than a biological sample not having high MN1. In some embodiments, the expression level of MN1 in a biological sample having “high MN1” is between about 50-fold and about 500-fold higher, for example between about 100-fold and about 500-fold higher than a biological sample not having high MN1. In some embodiments, the MN1 expression level of “high MN1” is between about 20-fold and about 3,500-fold higher than a biological sample not having high MN1. In some embodiments, the MN1 expression level of “high MN1” is at least 2-fold, at least 5-fold, at least 10-fold, at least 20-fold, at least 30-fold, at least 40-fold, at least 50-fold, at least 100-fold, at least 200-fold, at least 300-fold, at least 400-fold, at least 500-fold, at least 1,000-fold, at least 2,500-fold, or at least 5,000-fold higher than a biological sample not having high MN1.

In some embodiments, the expression level of HOXA9 in a biological sample having “high HOXA9” is between about 5-fold and about 5,000-fold higher than a biological sample not having high HOXA9. In some embodiments, the expression level of HOXA9 in a biological sample having “high HOXA9” is between about 10-fold and about 1,000-fold higher than a biological sample not having high HOXA9. In some embodiments, the expression level of HOXA9 in a biological sample having “high HOXA9” is between about 50-fold and about 500-fold higher, for example between about 100-fold and about 500-fold higher than a biological sample not having high HOXA9. In some embodiments, the HOXA9 expression level of “high HOXA9” is between about 20-fold and about 3,500-fold higher than a biological sample not having high HOXA9. In some embodiments, the HOXA9 expression level of “high HOXA9” is at least 5-fold, at least 10-fold, at least 20-fold, at least 30-fold, at least 40-fold, at least 50-fold, at least 100-fold, at least 200-fold, at least 300-fold, at least 400-fold, at least 500-fold, at least 1,000-fold, at least 2,500-fold, or at least 5,000-fold higher than a biological sample not having high HOXA9.

In some embodiments, DOT1L inhibitor compounds described herein inhibit the histone methyltransferase activity of DOT1L or a mutant thereof and are useful to treat certain forms of AML. Based upon the surprising discovery that methylation regulation by DOT1L is involved in progression of certain forms of AML, particular leukemia cells bearing an increased mRNA, protein and/or activity (function) level of at least MN1 and HOXA9 (and optionally MEIS1 and/or DOT1L), the compounds described herein are useful for treating certain forms of acute myeloid leukemia.

In some embodiments, the present invention features a method for treating or alleviating a symptom of MN1^{high}/HOXA9^{high} AML. The method includes administering to a subject in need thereof, a therapeutically effective amount of a compound of the present invention, or a pharmaceutically acceptable salt, prodrug, metabolite, polymorph, solvate, or stereoisomer thereof.

The present disclosure provides methods for the treatment of MN1^{high}/HOXA9^{high} AML mediated by DOT1 (e.g., DOT1L-mediated) protein methylation in a subject in need thereof by administering to a subject in need of such treatment, a therapeutically effective amount of a compound of the present invention (e.g., a DOT1L inhibitor), or a pharmaceutically acceptable salt, prodrug, metabolite, polymorph or solvate thereof. The present disclosure further provides the use of one or more DOT1L inhibitors, or a pharmaceutically acceptable salt, prodrug, metabolite, polymorph or solvate thereof, for the preparation of a medicament useful for the treatment of MN1^{high}/HOXA9^{high} AML mediated by DOT1L-mediated protein methylation.

In some embodiments, the present disclosure provides methods for the treatment of a MN1^{high}/HOXA9^{high} AML, the course of which is influenced by modulating the methylation status of histones or other proteins, wherein said methylation status is mediated at least in part by the activity of DOT1L.

Modulation of the methylation status of histones can in turn influence the level of expression of target genes activated by methylation, and/or target genes suppressed by methylation. The method includes administering to a subject in need of such treatment, a therapeutically effective amount of a DOT1L inhibitor as described herein, or a pharmaceutically acceptable salt, prodrug, metabolite, polymorph, solvate, or stereoisomer thereof.

In one aspect, methods described herein are useful to treat leukemia. In some embodiments, the leukemia is acute myeloid leukemia (AML). AML is a cancer of the myeloid line of blood cells characterized by the abnormal growth of white blood cells that accumulate in the bone marrow and interfere with the production of normal blood cells. AML has several subtypes. In some aspects, the instant disclosure relates to the subtype of AML associated with high MN1 and high HOXA9 (MN1^{high}/HOXA9^{high} AML). In some aspects, AML subtypes are associated with particular genetic lesions, including but not limited to balanced translocations, deletions, gene amplifications and aneuploidy. In some aspects, genetic lesions associated with AML subtypes are characterized by their cytogenetics. As used herein, “cytogenetics” refers to the chromosomal structure of a subject. Cytogenetic abnormalities, for example translocations or deletions, may be identified by a number of techniques known in the art, including but not limited to karyotyping, Fluorescence in situ hybridization (FISH), microarray-comparative genomic hybridization (CGH) and Next Generation Sequencing (NGS). In some aspects, cytogenetic abnormalities are associated with MN1^{high}/HOXA9^{high} AML. In some aspects, the cytogenetic abnormalities associated with MN1^{high}/HOXA9^{high} AML include but are not limited to del(5q) and del(7q). In some embodiments the cytogenetic abnormalities associated with MN1^{high}/HOXA9^{high} AML include del(5q), del(7q), or del(5q) and del(7q). As used herein, del(5q) and/or del(7q) refer to the presence of one or more deletions within the 5q and/or 7q chromosomal regions (the q arms of chromosomes 5 and 7 respectively). In some embodiments, del(5q) and/or del(7q) involve deletions of the entire 5q and/or 7q regions.

The present disclosure further provides the use of a compound described herein, or a pharmaceutically acceptable salt, ester, prodrug, metabolite, polymorph or solvate thereof in the treatment of MN1^{high}/HOXA9^{high} AML, or, for the preparation of a medicament useful for the treatment of such MN1^{high}/HOXA9^{high} AML.

Compounds of the present disclosure can selectively inhibit proliferation of leukemia cells characterized with an increased mRNA, protein and/or activity (function) level of at least MN1 and HOXA9 (and optionally MEIS1).

Accordingly, the present disclosure provides methods for treating or alleviating a symptom of MN1^{high}/HOXA9^{high} AML characterized with an increased mRNA, protein and/or activity (function) level of at least MN1 and HOXA9 proteins (and optionally MEIS1) by a compound of the present disclosure, or a pharmaceutically acceptable salt, ester, prodrug, metabolite, polymorph or solvate thereof.

The present disclosure also provides methods for treating or alleviating a symptom of MN1^{high}/HOXA9^{high} AML characterized by the presence of genetic lesions, for example del(5q) and del(7q). For example, in some embodiments a method comprises obtaining sample from a subject, detecting the presence of a genetic lesion associated with MN1^{high}/HOXA9^{high} AML (e.g., del(5q) and del(7q)) in the sample, and when the genetic lesion is present in the sample, administering to the subject a therapeutically effective amount of a DOT1L inhibitor

The present disclosure also provides methods for treating MN1^{high}/HOXA9^{high} AML mediated by deletion of chromosome 5 and/or chromosome 7, comprising administering to a subject in need thereof a therapeutically effective amount of a DOT1L inhibitor.

In other aspects, the present disclosure provides personalized medicine, treatment and/or AML management for a subject by genetic screening of increased gene expression (mRNA or protein), and/or increased function or activity level of at least one protein selected from the group consisting of MN1, HOXA9, and MEIS1 in the subject. For example, the present disclosure provides methods for treating, preventing or alleviating a symptom of leukemia or a precancerous condition by determining responsiveness of the subject to a DOT1L inhibitor and when the subject is responsive to the DOT1L inhibitor, administering to the subject a therapeutically effective amount of the DOT1L inhibitor, or a pharmaceutically acceptable salt, prodrug, metabolite, polymorph, solvate, or stereoisomer thereof. The responsiveness is determined by obtaining a sample from the subject and detecting increased mRNA or protein, and/or increased activity level of at least MN1 and HOXA9 (and optionally MEIS1), and the presence of such gain of expression and/or function indicates that the subject is responsive to the DOT1L inhibitor. Once the responsiveness of a subject is determined, a therapeutically effective amount of a DOT1L inhibitor can be administered. The therapeutically effective amount of a DOT1L inhibitor can be determined by one of ordinary skill in the art.

In other aspects, the present disclosure provides personalized medicine, treatment and/or cancer management for a subject by genetic screening of AML subtypes. In some aspects, AML subtypes are associated with particular genetic lesions, including but not limited to balanced translocations, deletions, gene amplifications and aneuploidy. In some aspects, genetic lesions associated with AML subtypes are characterized by their cytogenetics. As used herein, “cytogenetics” refers to the chromosomal structure of a subject. Cytogenetic abnormalities, for example translocations or deletions, may be identified by a number of techniques known in the art, including but not limited to karyotyping, Fluorescence in situ hybridization (FISH), microarray-comparative genomic hybridization (CGH) and Next Generation Sequencing (NGS). In some aspects, cytogenetic abnormalities are associated with MN1^{high}/HOXA9^{high} AML. In some embodiments, the cytogenetic abnormalities associated with MN1^{high}/HOXA9^{high} AML include but are not limited to del(5q) and del(7q). In some embodiments, the cytogenetic abnormalities associated with MN1^{high}/HOXA9^{high} AML include del(5q) and/or del(7q).

As used herein, the term “responsiveness” is interchangeable with terms “responsive”, “sensitive”, and “sensitivity”, and it is meant that a subject shows one or more therapeutic responses when administered an DOT1L inhibitor, e.g., leukemia cells or leukemia progenitor cells of the subject undergo apoptosis and/or necrosis, differentiation and/or display reduced growth, division, or proliferation. This term can also mean that a subject will or has a higher probability, relative to the population at large, of having a therapeutic response when administered an DOT1L inhibitor, e.g., leukemia cells or leukemia progenitor cells of the subject undergo apoptosis and/or necrosis, differentiation and/or display reduced growth, division, or proliferation.

As used herein, a “subject” is interchangeable with a “subject in need thereof”, both of which refers to a subject having a MN1^{high}/HOXA9^{high} AML that involves DOT1L-mediated protein methylation, or a subject having an increased risk of developing such a disorder relative to the population at large. A subject in need thereof may be a subject having a MN1^{high}/HOXA9^{high} AML. A subject in need thereof can have a precancerous condition. In some embodiments, a subject in need thereof has leukemia. A subject in need thereof can have leukemia associated with DOT1L, for example AML. A subject in need thereof can have AML associated with increased expression (mRNA or protein) and/or activity level of at least one MN1 and HOXA9 (and optionally MEIS1). A subject in need thereof can have MN1^{high}/HOXA9^{high} AML associated with the cytogenetic abnormalities del(5q) and del(7q).

As used herein, a “subject” includes a mammal. The mammal can be, e.g., a human or appropriate non-human mammal, such as a primate, mouse, rat, dog, cat, cow, horse, goat, camel, sheep or a pig. The subject can also be a bird or fowl. In one embodiment, the mammal is a human. A subject can be male or female.

A subject in need thereof can be one who has been previously diagnosed or identified as having leukemia or a precancerous condition. A subject in need thereof can also be one who is having (suffering from) leukemia or a precancerous condition. Alternatively, a subject in need thereof can be one who has an increased risk of developing such disorder relative to the population at large (e.g., a subject who is predisposed to developing such disorder relative to the population at large).

Optionally a subject in need thereof has already undergone, is undergoing or will undergo, at least one therapeutic intervention for the leukemia or precancerous condition.

A subject in need thereof may have refractory leukemia on most recent therapy.

“Refractory leukemia” means leukemia that does not respond to treatment. The leukemia may be resistant at the beginning of treatment or it may become resistant during treatment.

Refractory leukemia is also called resistant leukemia. In some embodiments, the subject in need thereof has leukemia recurrence following remission on most recent therapy. In some embodiments, the subject in need thereof received and failed all known effective therapies for cancer treatment. In some embodiments, the subject in need thereof received at least one prior therapy.

In some embodiments, a subject in need thereof may have a secondary leukemia as a result of a previous therapy. “Secondary leukemia” means leukemia that arises after, due to, or as a result from previous carcinogenic therapies, such as chemotherapy. In some embodiments, the secondary leukemia is AML. In some embodiments, the secondary leukemia is AML with del(5q) and/or del(7q). In some embodiments, the secondary leukemia is MN1^{high}/HOXA9^{high} AML.

In any method of the present disclosure, a subject in need thereof may have increased mRNA, protein, and/or activity level of at least signaling component downstream of at least one protein selected from the group consisting of MN1, HOXA9, and MEIS1. Such downstream components are readily known in the art, and can include other transcription factors, or signaling proteins.

As used herein, the terms “high”, “elevated”, or “increased” refer to increased amounts or a gain of function of a gene product/protein compared to the wild type. In one aspect of the present disclosure, increased activity can be caused by increased mRNA and/or increased protein levels. Increased mRNA levels can be caused by gene amplification and/or increased transcription, for example. Alternatively, in some embodiments, increased activity levels can be caused by a gain of function mutation resulting from a point mutation (e.g., a substitution, a missense mutation, or a nonsense mutation), an insertion, and/or a deletion, or a rearrangement in the polypeptide comprising MN1, HOXA9 or MEIS1, or the nucleic acid sequence encoding a polypeptide selected from the group consisting of MN1, HOXA9 or MEIS1, or a nucleic acid controlling the expression of a polypeptide selected from the group consisting of MN1, HOXA9 or MEIS1. In some embodiments, high MN1 and high HOXA9 are associated with chromosomal alterations (e.g., del(5q) and/or del(7q)).

In some embodiments, the mutations and/or chromosomal alterations referred to herein are somatic mutations or alterations. The term “somatic” mutation or alteration refers to a mutation or alteration (e.g., deleterious) in at least one gene allele (e.g., one or both alleles or copies of a chromosomal region) that is not found in every cell of the body, but is found only in isolated cells. A characteristic of the somatic changes as used herein is, that they are restricted to particular tissues or even parts of tissues or cells within a tissue and are not present in the whole organism harboring the tissues or cells. The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene.

Accordingly, an increase in mRNA or protein expression and/or activity levels can be detected using any suitable method available in the art. For example, an increase in activity level can be detected by measuring the biological function of a gene product (e.g., activity of MN1, HOXA9, or MEIS1), the transcriptional activity of MN1, HOXA9, or MEIS1, (e.g., expression levels of target genes can be assayed using RT-PCR or other suitable technique). In some embodiments, genetic modifications (e.g., one or more deletions of 5q and/or 7q chromosomal regions) are associated with increased expression of MN1 and HOXA9 (and optionally MEIS1) can be detected using a karyotype analysis, a hybridization (e.g., FISH or microarray-comparative genomic hybridization (CGH)) based analysis, and/or a sequencing analysis. In some embodiments, a gain of function mutation can be determined by detecting any alteration in a nucleic acid sequence encoding a protein selected from the group consisting of MN1, HOXA9 or MEIS1. For example, a nucleic acid sequence encoding MN1, HOXA9 or MEIS1 having a gain of function mutation can be detected by whole-genome resequencing or target region resequencing (the latter also known as targeted resequencing) using suitably selected sources of DNA and polymerase chain reaction (PCR) primers in accordance with methods well known in the art. Methods typically and generally entails the steps of genomic DNA purification, PCR amplification to amplify the region of interest, cycle sequencing, sequencing reaction cleanup, capillary electrophoresis, and/or data analysis. Alternatively or in addition, a method may include the use of microarray-based targeted region genomic DNA capture and/or sequencing. Kits, reagents, and methods for selecting appropriate PCR primers and performing resequencing are commercially available, for example, from Applied Biosystems, Agilent, and NimbleGen (Roche Diagnostics GmbH). Detection of mRNA expression can be detected by methods known in the art, such as Northern blot, nucleic acid PCR, quantitative RT-PCR, expression array or RNA-sequencing. Detection of polypeptide expression (e.g., wild-type or mutant) can be carried out with any suitable immunoassay in the art, such as Western blot analysis.

By “sample” is meant any biological sample derived from the subject, includes but is not limited to, cells, tissues samples, body fluids (including, but not limited to, mucus, blood, plasma, serum, urine, saliva, and semen), cancer cells, and cancer tissues. In some embodiments, the sample is selected from bone marrow, peripheral blood cells, blood, cerebrospinal fluid, skin lesions, chloroma biopsies, plasma and serum.

Samples can be provided by the subject under treatment or testing. Alternatively samples can be obtained by the physician according to routine practice in the art.

The present disclosure also provides methods for diagnosing leukemia in a subject by obtaining a sample from the subject and detecting an increased mRNA, protein and/or activity level of at least one protein selected from the group consisting of MN1, HOXA9, and MEIS1, and the presence of such increased mRNA, protein and/or activity level indicates that the subject has or is at risk for developing leukemia compared to a subject without such increased mRNA, protein and/or activity level, or a subject that does not have leukemia.

The present disclosure also provides methods for determining predisposition of a subject to MN1^{high}/HOXA9^{high} AML by obtaining a sample from the subject and detecting an increased mRNA, protein and/or activity level of at least one protein selected from the group consisting of MN1, HOXA9, and MEIS1, and the presence of such increased mRNA, protein and/or activity level indicates that the subject is predisposed to (e.g., has a higher risk of) developing leukemia compared to a subject without such increased mRNA, protein and/or activity level.

The term “predisposed” as used herein in relation to leukemia or a precancerous condition is to be understood to mean the increased probability (e.g., at least 1%, 5%, 10%, 20%, 30%, 40%, 50%, 60%, 70%, 80%, 90%, 100%, 150%, 200%, or more increase in probability) that a subject with an increased mRNA, protein and/or activity level of at least one protein selected from the group consisting of MN1, HOXA9, and MEIS1, will suffer leukemia, as compared to the probability that another subject not having an increased mRNA, protein and/or activity level of at least one protein selected from the group consisting of MN1, HOXA9, and MEIS1, will suffer leukemia, under circumstances where other risk factors (e.g., chemical/environment, food, and smoking history, etc.) for having leukemia between the subjects are the same.

“Risk” in the context of the present disclosure, relates to the probability that an event will occur over a specific time period and can mean a subject's “absolute” risk or “relative” risk. Absolute risk can be measured with reference to either actual observation post- measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion.

In other example, the present disclosure provides methods of AML management in a subject by determining predisposition of the subject to MN1^{high}/HOXA9^{high} AML periodically. The methods comprise steps of obtaining a sample from the subject and detecting increased mRNA or protein, and/or increased activity level of at least one protein selected from the group consisting of MN1, HOXA9, and MEIS1, and the presence of such gain of expression and/or function indicates that the subject is predisposed to developing MN1^{high}/HOXA9^{high} AML compared to a subject without such gain of mRNA or protein expression and/or function of the at least one protein selected from the group consisting of MN1, HOXA9, and MEIS1.

As used herein, the term “acute myeloid leukemia (AML)” refers to a cancer of the myeloid line of blood cells characterized by the abnormal growth of white blood cells that accumulate in the bone marrow and interfere with the production of normal blood cells. AML has several subtypes. In some aspects, the instant disclosure relates to the subtype of AML associated with high MN1 and high HOXA9 (MN1^{high}/HOXA9^{high} AML). In some aspects, AML subtypes are associated with particular genetic lesions, including but not limited to balanced translocations, deletions, gene amplifications and aneuploidy. In some aspects, genetic lesions associated with AML subtypes are characterized by their cytogenetics. As used herein, “cytogenetics” refers to the chromosomal structure of a subject. Cytogenetic abnormalities, for example translocations or deletions, may be identified by a number of techniques known in the art, including but not limited to karyotyping, Fluorescence in situ hybridization (FISH), microarray-comparative genomic hybridization (CGH) and Next Generation Sequencing (NGS). In some embodiments, cytogenetic abnormalities are associated with MN1^{high}/HOXA9^{high} AML. In some embodiments, the cytogenetic abnormalities associated with MN1^{high}/HOXA9^{high} AML include but are not limited to del(5q) and del(7q). In some embodiments, the del(5q) is an interstitial deletion, for example del(5)(q13q31), del(5)(q13q33), or del(5)(q22q33). In some embodiments, the del(7q) is an interstitial deletion, for instance with proximal breakpoints in bands q11-22 and distal breakpoints in q31-36 (e.g., del(7)(q22q35), del(7)(q21q34) or del(7)(q11q34)).

As used herein, “treating” or “treat” describes the management and care of a patient for the purpose of combating a disease, condition, or disorder and includes the administration of a compound of the present disclosure, or a pharmaceutically acceptable salt, prodrug, metabolite, polymorph or solvate thereof, to alleviate the symptoms or complications of a disease, condition or disorder, or to eliminate the disease, condition or disorder.

A compound of the present disclosure, or a pharmaceutically acceptable salt, prodrug, metabolite, polymorph or solvate thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as leukemia, which can occur in multiple locations, is alleviated if the severity of the leukemia is decreased within at least one of multiple locations.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals. As used herein the term “sign” is also defined as an indication that something is not right in the body. But signs are defined as things that can be seen by a doctor, nurse, or other health care professional.

Treating or preventing a leukemia can result in a reduction in the rate of leukemia cell or leukemia progenitor cell proliferation. Preferably, after treatment, the rate of leukemia-associated cell proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time. The rate of cellular proliferation may also be measured by any method commonly known in the art, for example flow cytometry.

Treating or preventing a leukemia can result in an increase in the rate of normal blood cell proliferation. Preferably, after treatment, the rate of normal blood cell proliferation is increased by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time. The rate of cellular proliferation may also be measured by any method commonly known in the art, for example flow cytometry.

Treating or preventing a leukemia can result in a reduction in the proportion of proliferating leukemia cells or leukemia progenitor cells. Preferably, after treatment, the proportion of proliferating leukemia cells or leukemia progenitor cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of non-dividing cells in a tissue sample. The proportion of proliferating cells can be equivalent to the mitotic index.

Treating or preventing a leukemia can result in an increase in the proportion of normal blood cells. Preferably, after treatment, the proportion of proliferating normal cells is increased by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating normal cells may be measured by any reproducible means of measurement. Preferably, the proportion of proliferating cells is measured, for example, by quantifying the number of dividing cells relative to the number of non-dividing cells in a tissue sample. The proportion of proliferating cells can be equivalent to the mitotic index.

Treating or preventing leukemia can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of excessive accumulation of immature cells (blasts) and differentiation arrest, or disordered (dysplastic) differentiation.

Treating leukemia, for example AML, can result in leukemia cell death, and preferably, leukemia cell death results in a decrease of at least 10% in number of leukemia cells in a population. More preferably, leukemia cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci U S A. 100(5): 2674-8, 2003. In an aspect, leukemia cell death occurs by apoptosis.

Treating leukemia, for example AML, can result in leukemia cell differentiation, and preferably, leukemia cell differentiation results in a decrease of at least 10% in number of undifferentiated leukemia cells (leukemic blasts) in a population. More preferably, leukemia cell differentiation means a decrease of at least 20%; more preferably, a decrease of at least 30%; more preferably, a decrease of at least 40%; more preferably, a decrease of at least 50%; most preferably, a decrease of at least 75%. The number of cells in a population may be measured by any reproducible means. The number of blasts and differentiated cells in a population can be measured by fluorescence activated cell sorting (FACS), immunofluorescence microscopy and light microscopy.

In some embodiments, an effective amount of a compound of the present disclosure, or a pharmaceutically acceptable salt, prodrug, metabolite, polymorph or solvate thereof, is not significantly cytotoxic to normal cells. A therapeutically effective amount of a compound is not significantly cytotoxic to normal cells if administration of the compound in a therapeutically effective amount does not induce normal cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the present disclosure, or a pharmaceutically acceptable salt, prodrug, metabolite, polymorph or solvate thereof, can induce or activate cell death selectively in AML cells. Administering to a subject in need thereof a compound of the present disclosure, or a pharmaceutically acceptable salt, prodrug, metabolite, polymorph or solvate thereof, can induce or activate cell death selectively in AML cells. Contacting a cell with a compound of the present disclosure, or a pharmaceutically acceptable salt, prodrug, metabolite, polymorph or solvate thereof, can induce cell death selectively in one or more cells affected by AML. Preferably, administering to a subject in need thereof a compound of the present disclosure, or a pharmaceutically acceptable salt, prodrug, metabolite, polymorph or solvate thereof, induces cell death selectively in one or more cells affected by AML.

In some embodiments, the present disclosure relates to a method of treating or preventing AML by administering a compound of the present disclosure, or a pharmaceutically acceptable salt, prodrug, metabolite, polymorph or solvate thereof, to a subject in need thereof, where administration of the compound of the present disclosure, or a pharmaceutically acceptable salt, prodrug, metabolite, polymorph or solvate thereof, results in one or more of the following: accumulation of cells in G1 and/or S phase of the cell cycle, cytotoxicity via cell death in AML cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N. Y.; Enna et al., Current Protocols in Pharmacology, John Wiley & Sons, N. Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

As used herein, a DOT1L inhibitor is an inhibitor of DOT1L-mediated protein methylation (e.g., an inhibitor of histone methylation). In some embodiments, a DOT1L inhibitor is a small molecule inhibitor of DOT1L. In some embodiments, a DOT1L inhibitor is a compound of formula:

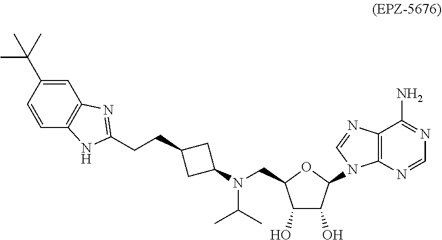 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, prodrug, metabolite, polymorph, solvate, or stereoisomer thereof.

In some embodiments, a DOT1L inhibitor is a compound of formula:

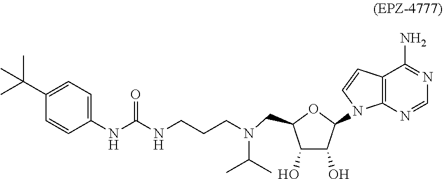 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, prodrug, metabolite, polymorph, solvate, or stereoisomer thereof.

Other DOT1L inhibitors suitable for use according to methods described herein are provided in WO2012/075381, WO2012/075492, WO2012/082436, WO2012/75500, WO2014/026198, WO2014/035140, and US2014/0100184, the contents of each of which are hereby incorporated by reference in their entirety. The activity of a DOT1L inhibitor can be evaluated in an assay, for example by comparing the histone methyltransferase activity of DOT1L (e.g., methylation of histone substrates such as H3K79 by immunoblot) in the presence or absence of different amounts of the inhibitor.

The disclosure also relates to a pharmaceutical composition of a therapeutically effective amount of a DOT1L inhibitor disclosed herein and a pharmaceutically acceptable carrier.

The disclosure also relates to a pharmaceutical composition of a therapeutically effective amount of a salt of a DOT1L inhibitor disclosed herein and a pharmaceutically acceptable carrier.

The disclosure also relates to a pharmaceutical composition of a therapeutically effective amount of a hydrate of a DOT1L inhibitor disclosed herein and a pharmaceutically acceptable carrier.

The present disclosure also relates to use of the compounds disclosed herein in preparation of a medicament for treating or preventing leukemia. The use includes a DOT1L inhibitor disclosed herein for administration to a subject in need thereof in a therapeutically effective amount. The leukemia can be AML. In some embodiments, the AML is MN1^{high}/HOXA9^{high} AML. In some embodiments, the MN1^{high}/HOXA9^{high} AML is associated with one or more deletions in 5q and/or 7q chromosomal regions. In some embodiments, the MN1^{high}/HOXA9^{high} AML is associated with one or more deletions of the 5q and/or 7q chromosomal region.

In some embodiments, compounds provided herein can be formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intraarterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered continuously (e.g., intravenously) three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations). In some embodiments the administration regimen is a continuous IV infusion (e.g., 24 hours per day) for one or more weeks (e.g., 1-4, 4-8, or longer), for example a 28-day continuous IV infusion of each 28-day cycle.

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It should be appreciated that in some embodiments, a DOT1L inhibitor compound or composition can be administered as a monotherapy. As used herein, “monotherapy” refers to the administration of a single active or therapeutic compound to a subject in need thereof. In some embodiments, monotherapy will involve administration of a therapeutically effective amount of a single active compound, for example, AML monotherapy with one of the DOT1L inhibitor compounds described herein, or a pharmaceutically acceptable salt, prodrug, metabolite, analog or derivative thereof, to a subject in need of treatment of AML. In one aspect, the single active DOT1L inhibitor compound is a compound described herein, or a pharmaceutically acceptable salt, prodrug, metabolite, polymorph or solvate thereof.

It will be appreciated that in some embodiments, two or more DOT1L inhibitor compounds can be administered to a subject (e.g., to treat AML).

It also will be appreciated that in some embodiments one or more DOT1L inhibitor compounds or compositions, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

In some embodiments, a DOT1L inhibitor compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U. S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, an additional therapeutically active agent is an AML standard of care agent. In certain embodiments, an additional therapeutically active agent is Ara-C, or daunorubicin. In certain embodiments, an additional therapeutically active agent is a DNA methyltransferase inhibitor. In certain embodiments, an additional therapeutically active agent is azacitidine or decitabine. In certain embodiments, an additional therapeutically active agent is a histone deacetylase inhibitor. In certain embodiments, an additional therapeutically active agent is vorinostat or panobinostat. In certain embodiments, an additional therapeutically active agent is a demethylase inhibitor. In certain embodiments, an additional therapeutically active agent is tranylcypromine or LSD1 inhibitor II. In certain embodiments, an additional therapeutically active agent is a bromodomain inhibitor. In certain embodiments, an additional therapeutically active agent is IBET-151 or JQ1. In certain embodiments, an additional therapeutically active agent is an ALL standard of care agent. In certain embodiments, an additional therapeutically active agent is mitoxantrone, methotrexate, mafosfamide, prednisolone, or vincristine.

In certain embodiments, an additional therapeutically active agent is prednisolone, dexamethasone, doxorubicin, vincristine, mafosfamide, cisplatin, carboplatin, Ara-C, rituximab, azacitadine, panobinostat, vorinostat, everolimus, rapamycin, ATRA (all-trans retinoic acid), daunorubicin, decitabine, Vidaza, mitoxantrone, or IBET-151.

It also should be appreciated that in some embodiments, a DOT1L inhibitor compound or composition can be administered in conjunction with chemotherapy, radiation therapy, and/or a cytostatic agent. In some embodiments, treatment methods described herein are administered in conjunction with anti-VEGF or anti-angiogenic factor, and/or p53 reactivation agent. Non-limiting examples of cancer chemotherapeutic agents include, but are not limited to, irinotecan (CPT-11); erlotinib; gefitinib (IressaTM); imatinib mesylate (Gleevec); oxalipatin; anthracyclins-idarubicin and daunorubicin; doxorubicin; alkylating agents such as melphalan and chlorambucil; cis-platinum, methotrexate, and alkaloids such as vindesine and vinblastine. A cytostatic agent is any agent capable of inhibiting or suppressing cellular growth and multiplication. Non-limiting examples of cytostatic agents include paclitaxel, 5-fluorouracil, 5-fluorouridine, mitomycin-C, doxorubicin, and zotarolimus. Other cancer therapeutics that can be used in conjunction with a DOT1L inhibitor include inhibitors of matrix metalloproteinases such as marimastat, growth factor antagonists, signal transduction inhibitors and protein kinase C inhibitors. In some embodiments, methods described herein can be used in combination with treatment options such immunotherapy and/or cancer vaccines.

It should be appreciated that in some embodiments, the term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies.

The present disclosure also provides pharmaceutical compositions comprising one or more DOT1L inhibitor compounds described herein, and optionally one or more additional agents described herein, in combination with at least one pharmaceutically acceptable excipient or carrier.

A “pharmaceutical composition” is a formulation containing one or more DOT1L inhibitor compounds in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition described herein can be administered to a subject in many of the well-known methods currently used for chemotherapeutic treatment. For example, for treatment of leukemia, a DOT1L inhibitor compound or formulation may be injected directly into the blood stream or body cavities or taken orally or applied through the skin with patches. The dose chosen should be sufficient to constitute effective treatment but not as high as to cause unacceptable side effects. The state of the disease condition (e.g., leukemia, for example, AML) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject' s body weight, size, and health; the nature and extent of the condition; and the therapeutic selected for administration. Therapeutically effective amounts for a given situation can be determined by routine experimentation that is within the skill and judgment of the clinician. In some embodiments, the disease or condition to be treated is leukemia (e.g., AML, for example MN1^{high}/HOXA9^{high} AML).

For a DOT1L inhibitor compound or formulation, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds described herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms described herein are dictated by and directly dependent on the unique characteristics of the active compound and the particular therapeutic effect to be achieved.

In therapeutic applications, the dosages of the pharmaceutical compositions used as described herein vary depending on the agent or combination of agents, the age, weight, and clinical condition of the recipient patient, and the experience and judgment of the clinician or practitioner administering the therapy, among other factors affecting the selected dosage. Generally, the dose should be sufficient to result in slowing, and preferably regressing, the proliferation of leukemia cells and also preferably causing complete regression of the leukemia. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m , and age in years). An effective amount of a pharmaceutical agent is that which provides an objectively identifiable improvement as noted by the clinician or other qualified observer. For example, regression of leukemia in a patient may be measured with reference to the number of leukemia cells or leukemia precursor cells. Decrease in the number of leukemia cells indicates regression. Regression is also indicated by failure of leukemia cells to reoccur after treatment has stopped. As used herein, the term “dosage effective manner” refers to amount of an active compound to produce the desired biological effect in a subject or cell.

The compounds of the present disclosure are capable of further forming salts.

As used herein, “pharmaceutically acceptable salts” refer to derivatives of the compounds described herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compounds described herein can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds described herein can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p 1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds can be found in Remington: the Science and Practice of Pharmacy, 19th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing aspects of the present disclosure. The examples do not limit the claimed invention. Based on the present disclosure the skilled artisan can identify and employ other components and methodology useful for practicing the present invention.

For the compounds described herein, compounds may be drawn with one particular configuration for simplicity. Such particular configurations are not to be construed as limiting the invention to one or another isomer, tautomer, regioisomer or stereoisomer, nor does it exclude mixtures of isomers, tautomers, regioisomers or stereoisomers.

The pharmaceutical compositions can be included in a container, pack, or dispenser together with instructions for administration. Also encompassed by the present disclosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable.

Moreover, two or more steps or actions can be conducted simultaneously.

Compounds suitable for the methods of the disclosure, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described herein.

To further assess a compound's drug-like properties, measurements of inhibition of cytochrome P450 enzymes and phase II metabolizing enzyme activity can also be measured either using recombinant human enzyme systems or more complex systems like human liver microsomes. Further, compounds can be assessed as substrates of these metabolic enzyme activities as well. These activities are useful in determining the potential of a compound to cause drug-drug interactions or generate metabolites that retain or have no useful antimicrobial activity.

To get an estimate of the potential of the compound to be orally bioavailable, one can also perform solubility and Caco-2 assays. The latter is a cell line from human epithelium that allows measurement of drug uptake and passage through a Caco-2 cell monolayer often growing within wells of a 24-well microtiter plate equipped with a 1 micron membrane. Free drug concentrations can be measured on the basolateral side of the monolayer, assessing the amount of drug that can pass through the intestinal monolayer. Appropriate controls to ensure monolayer integrity and tightness of gap junctions are needed. Using this same system one can get an estimate of P-glycoprotein mediated efflux. P-glycoprotein is a pump that localizes to the apical membrane of cells, forming polarized monolayers. This pump can abrogate the active or passive uptake across the Caco-2 cell membrane, resulting in less drug passing through the intestinal epithelial layer. These results are often done in conjunction with solubility measurements and both of these factors are known to contribute to oral bioavailability in mammals. Measurements of oral bioavailability in animals and ultimately in man using traditional pharmacokinetic experiments will determine the absolute oral bioavailability.

Experimental results can also be used to build models that help predict physical-chemical parameters that contribute to drug-like properties. When such a model is verified, experimental methodology can be reduced, with increased reliance on the model predictability.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples are meant to illustrate, but in no way to limit, the claimed invention.

### Example 1

### Materials and Methods

### PCR and Primers

Dot1L Genotyping was performed using primers p2 (CCCAAAAGGGTCTTTTCACA, forward (SEQ ID NO:1)) and p4 (CACAGAGCCATGACCAGACA, reverse (SEQ ID NO:2)). Excision is confirmed using primers p1 (CTCACAGTCACATACTACCTCTGAC, forward (SEQ ID NO:3)) and p3 (ATGGGATTTCATGGAAGCAA, reverse (SEQ ID NO:4)) for the excised allele, and p2 and p3 for the floxed allele.

### Generation of MN-1 Leukemias

The MN-1 cDNA was re-cloned into an MSCV based vector (MIG, MSCV-IRES-GFP) followed by IRES-GFP cassette (MN1-GFP). The MSCV-based Cre-IRES-pTomato (cre) and MSCV-IRES-pTomato (control) or MSCV-based Cre-IRES-trCD2 (cre) and MSCV-IRES-CD2 (control) were cloned by inserting the cDNA or either pTomato or truncated human CD2 in place of GFP in MIG.

### Ecotropic Retroviral Vectors

MN-1-IRES-GFP, Cre-IRES-pTomato, Cre-IRES-trCD2, MSCV-IRES-pTomato or MSCV-IRES-CD2 were generated by cotransfection of 293T cells using FuGENE6 (Roche Molecular Biochemicals, Indianapolis, Ind.). Virus containing supernatant medium was collected on days 2 and 3 days after the transfection. Dotir bone marrow cell suspensions were prepared by crushing bones in a mortar after removal of muscle and connective tissues. Red blood cells were lysed on ice using red blood cells lysis buffer Pharm Lyse (BD Biosciences). Lineage depletion was performed by labeling bone marrow cell suspensions with a mixture of purified biotinylated monoclonal antibodies to CD3e (17A2), CD4 (GK1. 5), CD8a (53. 6. 7), CD19 (1D3), B220 (RA3. 6B2), Gr-1 (RB6. 8C5), IL-7R (A7R34) and Ter-119 (eBioscience, San Diego, Calif.). Lin+ cells were partially removed by 2 rounds of magnetic bead depletion with streptavidin conjugated Dynabeads (Dynal, Life Technologies, Carlsbad, Calif.). SLAM, LT-HSC, LSK or CMP cells were prepared by staining lineage depleted (lin) cells with APC-Cy7 conjugated streptavidin (Molecular Probes, Life Technologies, Carlsbad, Calif.) and stained with combinations of, c-Kit Alexa 647 (clone 2B8), CD48 Pacific Blue (clone HM48-1), CD150 PE (SLAM, clone TC15-12F12. 2), CD135 PE (Flk2, clone A2F10), CD16/32 PE (FcγRII, clone 93) all BioLegend (San Diego, Calif.), CD34 FITC (clone RAM34) BD Pharmingen (San Jose, Calif.), Sca-1 PE-Cy7 (clone D7) eBioscience (Affymetrix, San Diego, Calif.), and sorted for Lin⁻ Sca-1⁺c-Kit⁺ CD48⁻CD150⁺ (SLAM), Lin⁻Sca-1⁺ c-Kit⁺CD34⁻CD135⁻ (LT-HSC), Lin⁻Sca-1⁺ c-Kit⁺ (LSK) or Lin⁻Sca-1⁻c-Kit⁺CD34⁺CD32^{low} (CMP). Sorted cells were pre-stimulated for 24 h with 10 ng/ml mIL3 and mIL6 and 20 ng/ml mSCF, mF1t3L and TPO (Peprotec, Rocky Hill, N.J.). Transduction was carried out on retronectin (Takara, Madison, Wis.) with MN1-GFP in the presence of murine IL3, IL6, SCF, Flt3L and TPO in concentrations as above. Cells were subsequently maintained in M3234 (Stem cell technologies, Vancouver, BC) methylcellulose with 10 ng/ml IL3 and IL6, 20 ng/ml SCF, and 50 U/ml Penicillin/Streptomycin (Gibco, Life Technologies, Carlsbad, Calif.).

After 4-6 days, GFP-expressing cells were sorted and transduced with cre or control vector as described above. Four days after transduction, GFP⁺/pTomato⁺ or GFP⁺/CD2⁺ cells were sorted and transplanted into 6 week old C57BL/6 female irradiated (750 RAD) recipients at 1×10⁵ cells/mouse. 1×10⁵ cells/mouse of normal bone marrow were co-transplanted for early support. For secondary transplants, whole bone marrow from moribund leukemic mice was isolated, GFP⁺ cells were sorted and blast colonies were allowed to grow out in M3234 as described above. Leukemic cells were transduced with cre or control vector, sorted and transplanted as described above. Cell sorting was performed on a Beckman-Coulter MoFlo XDP70, MoFlo AstriosEQ or Beckton-Dickinson Aria IIu cell sorter.

Cell Growth assays for murine MN1 AML

For colony assays, sorted transduced leukemia cells were plated in methylcellulose M3234 containing IL3, IL6 and SCF at 1000 cells per plate in duplicate, and replated weekly at 500 cells/plate. Dot1l deletion was verified by PCR at each replating. For liquid culture, cells were maintained in media containing IL3, IL6 and SCF, and counted and replated at equal densities every 3-4 days.

Biochemical Assays (Cell Growth, Apoptosis, Cell Cycle Analysis, Western Blotting, qPCR)

Cell growth and viability were followed by serial cell counts and trypan blue exclusion staining. For colony assays, sorted transduced cells were plated in methylcellulose M3234 containing 10 ng/ml IL3 and IL6, and 20 ng/ml SCF at a concentration of 1000 or 5000 cells per plate, and replated at 1000 cells/plate every 6-7 days.

For the Annexin V apoptosis assay, 1×10⁶ cells were washed in PBS, resuspended in Ca/HEPES buffer (10 mM HEPES, pH7. 4; 140 mM NaCl; 2. 5 mM CaCl2) and incubated with Annexin V-APC (BD Pharmingen, San Jose, Calif.) for 30 min. DAPI (Molecular Probes, Life Technologies, Carlsbad, Calif.) was added prior to analysis.

Cell cycle analysis was performed after EdU labeling for 30 minutes using the EdU-Alexa647 kit from Molecular Probes (Life Technologies, Carlsbad, Calif.) according to the manufacturer's instructions and DAPI added prior to analysis. Data was acquired on a Beckton-Dickinson Gallios 561 cytometer and analyzed using Kaluza™ (Beckton-Dickinson).

For western blotting, histones were extracted by (triton extraction (PBS 0.5% TritonX100 (v/v), 2 mM phenylmethylsulfonylfluoride, 0.02% (w/v) NaN3 acid extraction) followed by acid extraction with 0.2N HCl. Proteins were separated on a 10% Bis-Tris gel (Nupage, Life Technologies, Carlsbad, Calif.) and blotted on nitrocellulose membranes (Novex, Life Technologies, Carlsbad, Calif.). The following antibodies were used for detection: H3K79me2 rabbit polyclonal abcam (Cambridge, Mass.) 3594-100, total H3 rabbit polyclonal abcam 1791; secondary antibody for detection: donkey anti rabbit ECL horseradish peroxidase linked NA934V, GE healthcare UK limited (Little Chalfont Buckinghamshire, UK). Proteins were visualized using Western Lightning Plus-ECL (Perkin-Elmer).

For reverse transcription and quantitative PCR, total RNA was isolated using Trizol (Life Technologies, Carlsbad, Calif.) or RNeasy mini or micro kit (Qiagen, Hilden, Germany) according to the manufacturer's instruction. Resultant cDNA was generated using the tetro cDNA synthesis kit (Bioline, Taunton, Mass.). Real time PCR was performed using TaqMan detection reagents (TaqMan Gene Expression Master Mix, Applied Biosystems) on the StepOnePlus Real-Time PCR System (Applied Biosystems) using TaqMan probes (all Life Technologies) for HoxA9 (Mm00439364_ml), Meis1a (Mm00487664_ml) and MN1 (Hs00159202_m1). The data were normalized to GAPDH (Mm99999915_gl) and are presented as fold change with respect to cells transduced with vector control. All experiments were performed with three technical replicates from two to four individual experiments.

### RNA-Sequencing

RNA was isolated from sorted GFP⁺/pTomato⁺ cells using Trizol (Invitrogen), or RNAeasy mini or micro kit (Qiagen, Hilden, Germany). cDNA libraries were constructed for each sample (3 cre, 3 MIT) using the Illumina TruSeq Stranded mRNA sample preparation kit (Illumina, San Diego, Calif.). The six independently and uniquely indexed libraries were pooled and loaded onto a single lane of a HiSeq2000 flowcell for single-end, 50-bp DNA sequencing using an Illumina HiSeq2000.

### Data Analysis and Statistical Methods.

NextGen RNAseq of six cDNA libraries yielded 29. 2 to 76. 8 million total reads per sample. Removal of low-quality bases [Phred score <15] using a custom Python script reduced total sequence data by ≤4%. The remaining sequences were mapped to the annotated mm9 genome (Dumas; NCBI NC_001348) using GSNAP. The bi-directional, strand-specific cDNA library construction protocol permitted alignment of sequences to either the annotated (top strand) or the complementary (bottom strand) of the mm9 genome using CUFFLINKS. After strand alignment of mm9 sequences, the fragments per kilobase of exon per million mapped reads was determined. FPKMs from all six libraries were analyzed using the statistical transformation technique of principal components analysis (PCA) to visualize the differences between samples. Samples were first separated by the largest component of variance (principal component 1, PC1), followed by separation of the next largest and independent component of variance (PC2). Differential gene expression determined using ANOVA, graphic representation at p<0.01 is shown as a heat map. The Dot1l-dependent in ^{MN1}CMP signature was determined as the top 200 differentially expressed genes. Gene set enrichment analysis (GSEA) was performed using (www.broadinstitute.org/gsea).

### Human Samples:

The samples from AML patients were obtained from initial diagnostic procedures at the University of Colorado Hospital (Protocol 06-0720), with patient informed consent for genetic analysis according to the Declaration of Helsinki, and institutional review board approval from all participating centers. Phenotypic analysis, conventional chromosome banding and fluorescence-in-situ-hybridization (FISH) were performed as previously described. Molecular analysis was performed at Children's Hospital Colorado, Department of Pathology as previously described. The samples included into the study contained at least 80% of leukemic cells following Ficoll-density gradient centrifugation based enrichment.

qPCR Analysis of Human HOXA9 and MN1 in AML Patient Samples

Expression of MN1 and HOXA9 was determined using Taq-man primer/probes. Fold-change of MN1 compared to normal CD33+ myeloid progenitors from 2 normal volunteers was calculated using the delta-CT method. HOXA9 is not expressed in normal CD33+ myeloid progenitors. Fold-change of HOXA9 was calculated compared to MLL-rearranged AML25 using the delta-CT method.

### Cell Growth Assays and DOT1L Inhibition for Patient Samples

Patient AML samples were plated in media containing TPO, Flt3L, IL3, IL6 and SCF as described by (Klco et al., 2013). Samples were plated on a small array of feeder cells (OP9, HS27, HS27a, AFT024) to determine optimal growth support. The DOT1L inhibitor EPZ4777 or DMSO control was added at the indicated concentrations. Inhibition of H3K79 methylation was verified by Western Blotting on day 4. Cells counted, washed and replated in fresh compound at equal densities every 3-4 days for 10-21 days.

### Dot1l Knockout Mice, Breeding

Animals were maintained at the Animal Research Facility at the University of Colorado Anschutz Medical Campus. Animal experiments were approved by the Internal Animal Care and Use Committee. Dot1l conditional knockout mice were previously described and were maintained on a fully backcrossed C57BL/6 background.

### Generation of Transformed Murine Cells and Leukemia

Ecotropic retroviral vectors containing murine MN1-IRES-GFP, Cre-IRES-pTomato (Cre) and MSCV-IRES-pTomato (MIT) were generated by cotransfection of 293 cells. Lin⁻Sca-1⁺ c-Kit⁺CD48⁻CD150⁺ (SLAM) , Lin⁻Sca-1⁺ c-Kit⁺CD34⁻Flk2⁻ (LT-HSC), Lin⁻Sca-1⁺ c-Kit⁺ (LSC) or Lin⁻Sca-1⁻ c-Kit⁺CD34⁺ FcgammaR^{low} (CMP) cells were transduced with MN1-GFP and maintained with supplemental cytokines. After 2-7 days, GFP⁺ cells were sorted and transduced with Cre or MIT. 2-3 days after transduction, GFP⁺/pTomato⁺ cells were sorted and transplanted into C57BL/6 syngeneic irradiated (750 rad) recipients at 1×10⁵ cells/mouse with syngenetic support marrow. For secondary transplants, whole bone marrow from leukemic mice was isolated, GFP⁺ cells were sorted and blast colonies were allowed to grow out. Leukemic cells were transduced with Cre or MIT, sorted and transplanted as described above.

Biochemical Assays (Apoptosis, Cell Cycle Aanalysis, Western Blotting, qPCR)

Cell growth and viability were followed by serial cell counts. Apoptosis and cell cycle analysis were performed using the Annexin-staining from BD-Pharmingen (San Jose, Calif.), and Click-IT EdU kit from Molecular Probes/Life Technologies (Grand Island, N.Y.). Antibodies used for flow cytometry and immunoblot detection and qPCR primers are detailed in the above methods.

### Cell Growth Assays for Murine MN1 AML

For colony assays, sorted transduced leukemia cells were plated in methylcellulose M3234 containing IL3, IL6 and SCF at 1000 cells per plate in duplicate, and replated weekly at 500 cells/plate. Dotll deletion was verified by PCR at each replating. For liquid culture, cells were maintained in media containing IL3, IL6 and SCF, and counted and replated at equal densities every 3-4 days.

### Cell Growth Assays and DOT1L Inhibition for Patient Samples

Patient AML samples were plated in media containing TPO, Flt3L, IL3, IL6 and SCF as described by (Klco et al., 2013). Samples were plated on a small array of feeder cells (OP9, HS27, HS27a, AFT024) to determine optimal growth support. The DOT1L inhibitor EPZ4777 or DMSO control was added at the indicated concentrations. Inhibition of H3K79 methylation was verified by Western Blotting on day 4. Cells counted, washed and replated in fresh compound at equal densities every 3-4 days for 10-21 days.

### RNA Amplification and Gene Expression Array

RNA was isolated from 10⁵ sorted GFP⁺/pTomato⁺ cells using Trizol (Invitrogen), or RNAeasy mini columns (Qiagen), and submitted to the UC-Denver genomics core for library preparation and sequencing.

### Data Analysis and Statistical Methods

Raw sequences obtained from RNA-Sequencing were trimmed and mapped to mm9 using GSNAP. Gene expression was calculated using CUFFLINKS, differential gene expression was determined using ANOVA. Gene set enrichment analysis (GSEA) was performed using (www. broadinstitute. org/gsea). RNA-Sequencing data has been deposited at the NCBI Gene Expression Omnibus (www.ncbi.nlm.nih.gov.ezp-prodl.hul.harvard.edu/geo/).

### Example 2

### Loss of Dot1l in Normal Early Hematopoietic Progenitors Leads to Down-Regulation of a Distinct Gene Expression Program

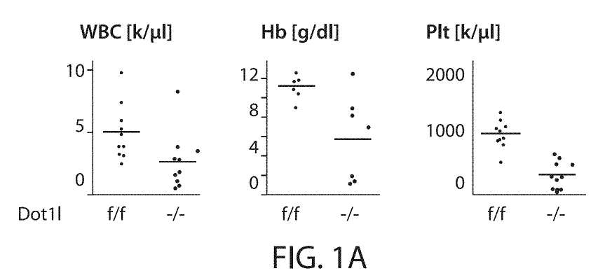

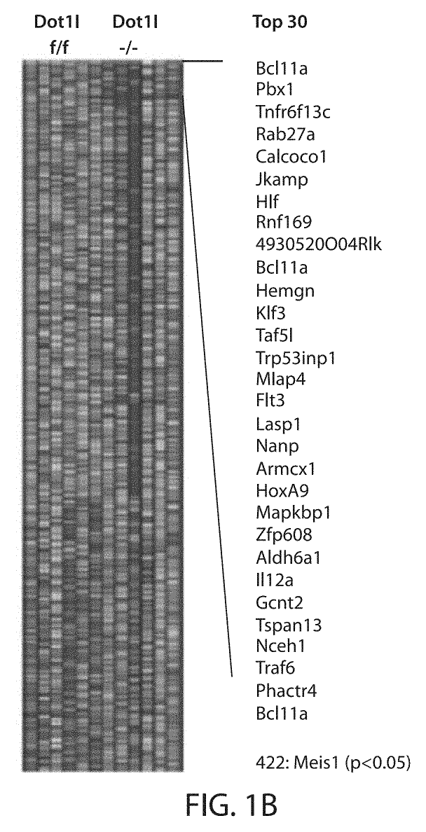

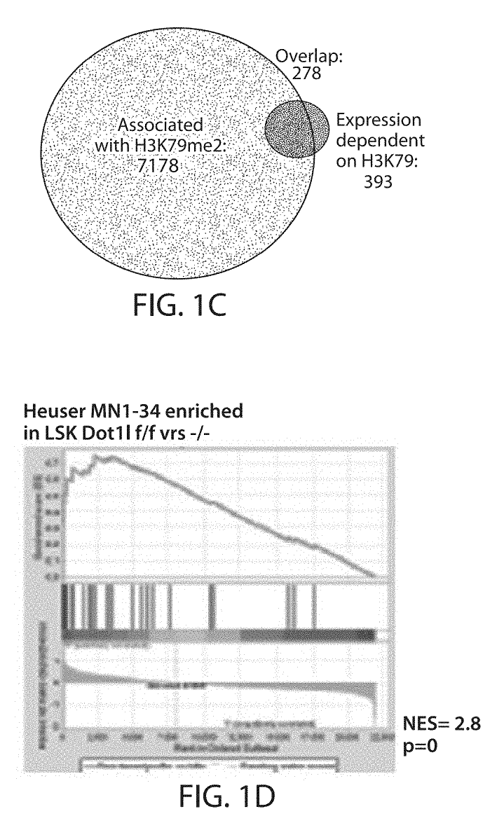

To delineate early gene expression changes that occur after genetic inactivation of Dot1l in normal hematopoiesis, conditional Dot1l^{f/f} mice were crossed into the MxCre model, which allows rapid and precise excision of exon 5 of the Dot1l gene (which contains most of the active site) after two doses of pI:pC. Induced Dot1lf/f-MxCre mice developed pancytopenia similar to previously reported for conditional Dot1l inactivation models using Tamoxifen inducible systems (FIG. 1A, (Jo et al., 2011; Nguyen et al., 2011)). In the Mx-Cre model, loss of functional Dot1l was confined to the hematopoietic system, and the high efficiency of the MxCre promoter allowed analysis of cell autonomous gene expression changes at a defined early time point. lin- Sca-1+ cKit+ (LSK) cells were isolated 6 days after pI:pC injection. The interferon response elicited by pI:pC treatment has been shown to lead to a temporary loss of quiescence in the hematopoietic stem cell (HSC) compartment, and distorts the ability to isolate HSC/progenitors using flow cytometric markers. However, these effects are resolved after 5 days (Essers et al., 2009), and flow cytometric analysis of pI:pC injected animals performed 6 days after the last dose shows a clearly distinguishable LSK population (FIG. 8A). Gene expression analyses were performed comparing pI:pC injected Dot lIff-MxCre LSK cells (Dot1l^{-/-}) to LSK cells from pI:pC injected Dot1l^{f/f} littermates (Dot1l^{f/f}). Similar to results that were previously reported in MLL-rearranged leukemias, loss of Dot1l led to down-regulation of a specific set of genes without inducing global transcriptional changes (393 genes at p=0.05, FIG. 1B and FIG. 1C). The set of genes whose expression was decreased in Dot1l^{-/-} LSK cells were defined as “Dot1l-dependent in LSK”. As expected, the majority of the “Dot1l-dependent in LSK” genes were associated with high levels of H3K79 dimethylation downstream of the transcription start site (as determined by ChIP-Seq in (Bernt et al., 2011), FIG. 1C). Gene set enrichment analysis indicated that this gene-set has significant overlap with genes regulated by Dot1l in MLL-rearranged leukemias, including the key MLL-fusion downstream target genes HoxA9 and Meis1 (FIG. 1B and 8B). In addition, many of these genes are down-regulated at the LSK/CPM to GMP transition (FIG. 8C).

### Example 3

### The Meningeoma-1 (MN1) Cooperating Gene Expression Program is Dependent on Functional Dot1l in LSK Cells

Heuser et al. (Heuser et al., 2011) reported that a specific, cell of origin derived gene expression program in common myeloid progenitors (CMPs) cooperates with overexpressed Meningeoma 1 (MN1) to cause myeloid leukemia. HoxA9 and Meis1 were identified as key components of this program, and the developmental transcriptional down-regulation at the transition to GMP appears similar to the Dotll-dependent program defined in FIG. 1B. The instant example therefore asks whether this cell-of-origin derived, MN1-cooperating gene expression program is dependent on Dotll in normal early hematopoietic progenitors. Indeed, gene set enrichment analysis demonstrated a strong enrichment of the “Dot1l-dependent in LSK” gene set in the gene expression program that defined MN1 leukemias in the work of Heuser et al. (FIG. 1D).

### Example 4

### MN1 Induced CMP Derived AML is Dependent on Functional Dot1l

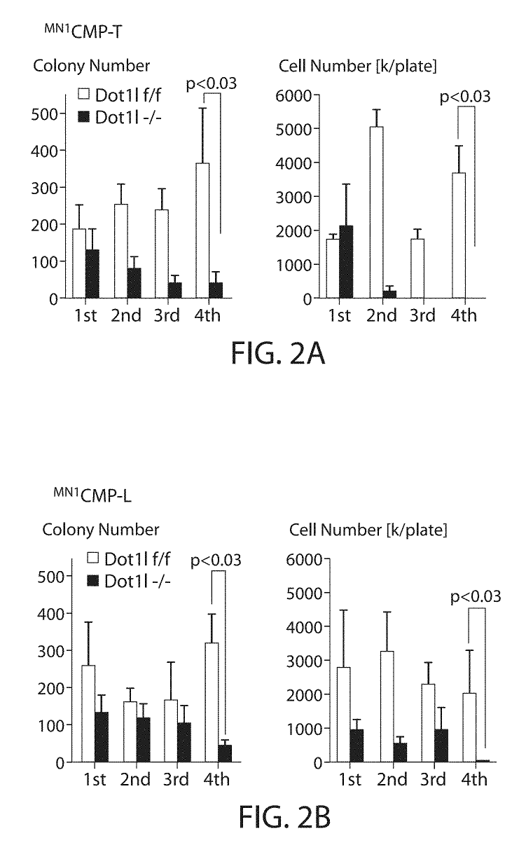

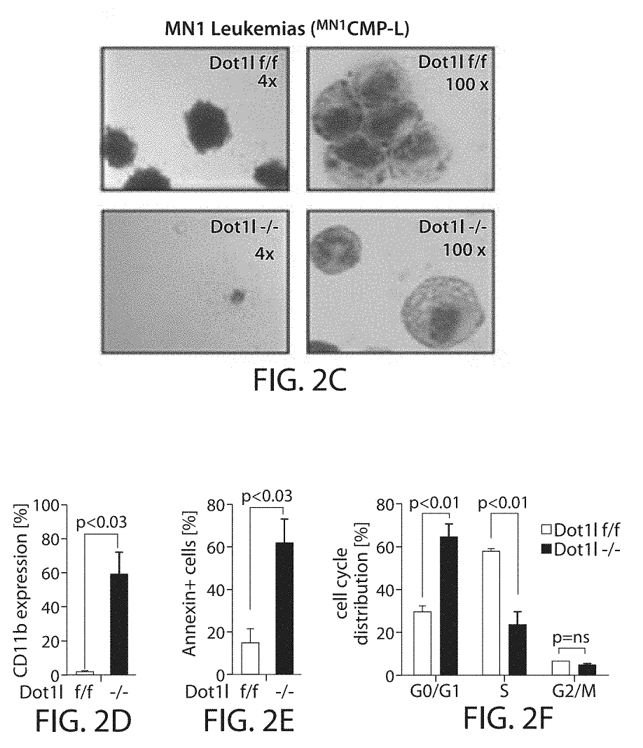

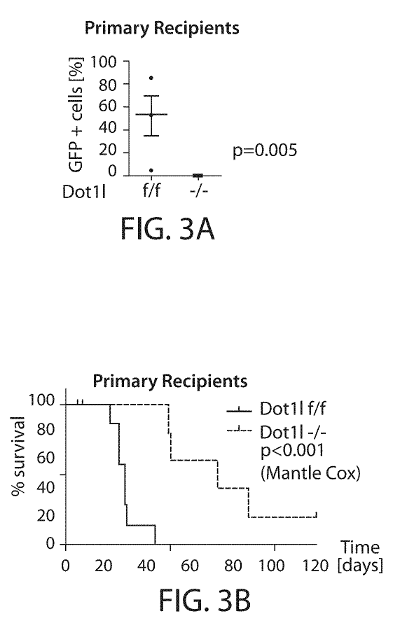

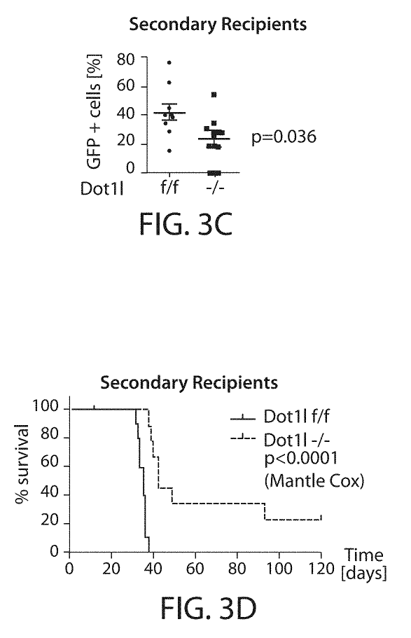

HoxA9/Meis 1 expression in the cell of origin is critically important for the ability of MN1 to induce AML (Heuser et al., 2011). Based on results showing that HoxA9 and Meis1 expression are dependent on functional Dot1l in early hematopoietic progenitors, the present work studies whether this dependency on Dot1l is preserved in MN1 leukemias. The human MN1 cDNA was introduced into sorted Dot1l^{f/f} CMP to establish in vitro transformed ^{MN1}CMP-T. Deletion of Dot1l through introduction of Cre (Dot1l^{-/- MN1}CMP-T) resulted in reduced cell numbers and colonies in serial replating assays (FIG. 2A and FIG. 2B). MN1 transduced Dot1l^{f/f} CMPs was also injected into recipient mice to establish Dot1l-conditional leukemias (mmCMP-L). Excision of exon 5 of Dot1l in ^{MN1}CMP-L isolated from moribund mice again resulted in decreased replating efficiency and decreased cell numbers (FIG. 2B). Dot1l^{-/-} colonies were smaller (FIG. 2C), and Dot1l^{-/-} MN1CMP-Leukemia cells showed morphologic signs consistent with increased differentiation. This was reflected in an increase in the expression of the myeloid differentiation marker CD11b in Dot1l^{-/- MN1}CMP-L (FIG. 2D). Loss of Dot1l also resulted in increased spontaneous apoptosis (FIG. 2E), and a decrease in the fraction of cycling cells (FIG. 2F). Similar results were observed in Dot1l^{-/- MN1}CMP-T cells (FIG. 9A-C). While the smaller colony size and increased differentiation mimic the effect of loss of Dot1l in MLL-rearranged leukemias, several subtle differences were observed between the two models. Loss of Dot1l in MLL-rearranged leukemias causes a minimal increase in apoptosis, while apoptosis in Dot1l^{-/-} MN1 was more pronounced. More importantly, while isolate viable and proliferating Dot1l^{-/-} MLL-AF9 cells were not able to be isolated beyond the third replating, serial replating of Dot1l^{-/- MN}1 leukemias was inefficient, but possible. Next, the effect of loss of functional Dot1l on in vivo leukemias was analyzed. Dot1l^{f/f MN1}CMP-L were transduced with Cre and injected into secondary recipients. While control mice succumbed to leukemia within 3-4 weeks, Dot1l^{-/- MN1}CMP-L injected animals experienced significantly decreased leukemic burden (FIG. 3A) and prolonged survival (FIG. 3B). All leukemias that eventually did develop in this cohort were found to be at least heterozygous for the floxed Dot1l allele by genotyping (FIG. 10) and had thus escaped full genetic inactivation of Dot1l.

### Example 5

### Loss of Dot1l Leads to Down-Regulation of the MN1-Cooperating Program in MN1 Transformed CMPs

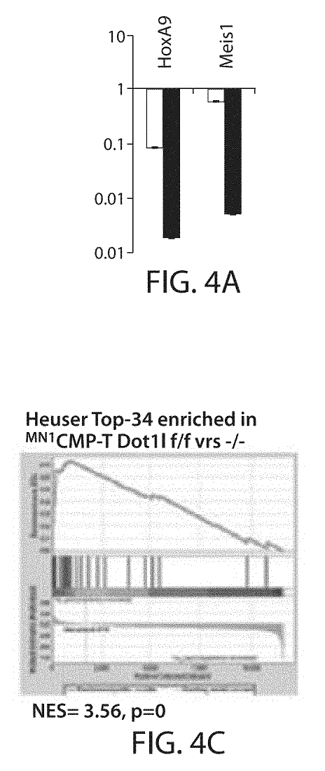

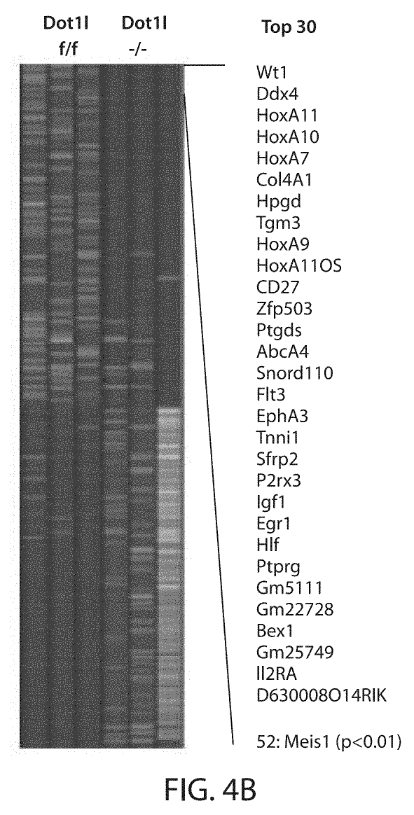

Next the question of whether the loss of functional Dot1l in CMP derived MN1 transformed cells resulted in down-regulation of the MN1-cooperating program defined by Heuser et al., including the key loci HoxA9 and Meisl was studied. qPCR analysis of ^{MN1}CMP-T 7 and 21 days after excision of exon 5 of Dot1l was performed, and found persistent down-regulation of HoxA9 and Meisl (FIG. 4A). In order to evaluate gene expression changes on a whole transcriptome scale, RNA-Seq of ^{MN1}CMP-T was performed 7 days after introduction of Cre. Similar to loss of Dot1l in other model systems, a defined gene set was found to have decreased expression after loss of Dot1l (FIG. 4B). HoxA9, HoxA10 and Meisl were among the most dysregulated genes. The MN1 cooperating program defined by Heuser et al (“Heuser Top-34”) showed significant enrichment in Dot1lf/f versus −/− ^{MN1}CMP-Ts, suggesting dependence of this program on functional Dot1l (FIG. 4C). Enrichment of the genes dependent on Dot1l in ^{MN1}CMP (“Down in ^{MN1}CMP-T Dot1−/−”) was also assessed in normal LSK versus GMP, LSK Dot1l^{f/f} versus Dot1l^{-/-} and MLL-AF9 leukemia Dot1l^{f/f} versus Dot1l^{-/-} data sets (FIG. 11A-C): enrichment was found in normal LSK (versus GMP or Dot1l−/− LSK) and Dot1lf/f MLL-AF9 leukemias (versus Dot1l−/−).

### Example 6

### Hematopoietic Stem Cells are an Inferior Cell of Origin for MN1 Driven, but not MLL-AF9 Driven AML

Isolation of CMPs for transduction with MN1 in the experiments described above was based on published results indicating transformed CMPs are the most efficient cell of origin in this model: MN1-transduced CMPs readily caused leukemia in recipient mice, while MN1-transduced hematopoietic stem cells (HSC) did not (Heuser et al., 2011). Despite their inability to cause leukemia in mice, MN1 transduced HSC were able to serially replate. This was attributed to a lower expression of HoxA9 in the HSC compartment compared to CMPs enough to allow in vitro immortalization, but not enough to cause leukemia in an in vivo model. (Heuser et al., 2011). The lower level of HoxA9 expression in HSC-derived MN1 transformed cells may reflect the endogenous regulation of the HoxA cluster in normal HSCs: a recently published RNA-Seq data set comparing transcriptional programs in adult and embryonic early hematopoiesis reports lower expression levels of HoxA9 in small numbers of highly purified HSC compared to LMPPs (FIG. 12F, (Boiers et al., 2013)).

The anti-leukemic effect of deletion of Dot1l in ^{MN1}CMP-T appears to be mediated by modulating a specific gene expression program in normal CMPs, which cooperates with MN1. However, the cell of origin in human MN1-high AML is not known, and may be variable in patients. Determining whether the dependence on Dotll is preserved if cells at an earlier stage of hematopoietic development serve as cell of origin may therefore have implications for the clinical translation of this data. Since MN1 transduced HSCs proliferate in vitro, at least an in vitro assessment of Dotll dependence is possible. As a first step, confirmation that HSCs are indeed incapable of serving as cell of origin in the murine model was sought. In addition, HoxA9 and Meisl expression levels in HSC-derived MN1 transformed cells (^{MN1}HSC-T), and their dependence on Dotll were investigated.

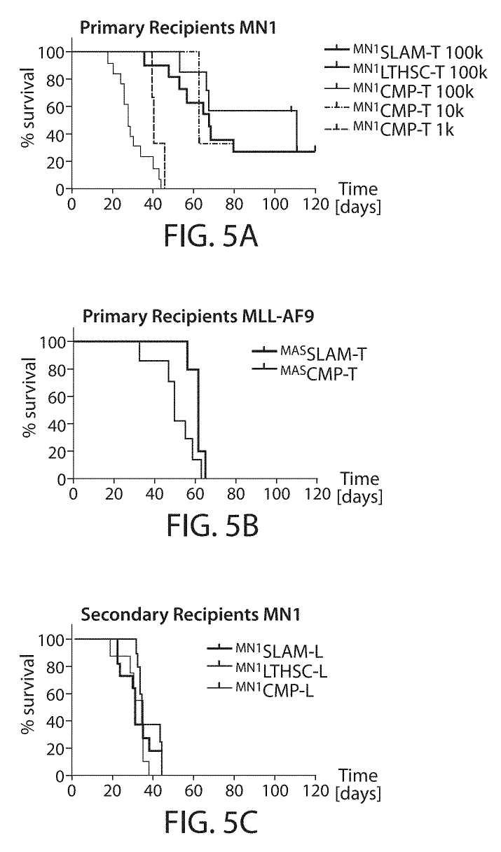

HSC-enriched populations were isolated from donor mice using two well established flow cytometric approaches, LT-HSCs and LSK-SLAM. Both strategies have been shown to yield a population that is highly enriched for functional hematopoietic stem cells. Mice injected with 100,000 ^{MN1}HSC-T (LT-HSC or LSK-SLAM) did develop leukemia, but with a longer latency, and with incomplete penetrance (FIG. 5A). To better compare the relative leukemia initiating cell frequency between CMP and HSC derived MN1 leukemias, limiting dilution experiments were performed with CMP derived MN1 transduced cells in parallel. LIC frequency in ^{MN1}HSC-T was 100-fold lower than in ^{MN1}CMP-T (FIG. 5A), confirming that HSCs are inferior to CMPs as cell of origin for MN1-driven AML. In fact, given that the starting populations for HSC transductions were highly enriched for HSCs, but not pure, the possibility that some or all of these leukemias originated from a co-purified early progenitor could not be excluded. This phenotype is specific to MN1-transduced HSC: when MLL-AF9 was introduced into CMPs and HSCs, no overt differences in latency or penetrance were seen (FIG. 5B), consistent with previously published results in this model (Krivtsov et al., 2013). In fact, Krivtsov et al. described an increased LIC frequency and more chemotherapy resistant disease when using LT-HSCs as cell of origin for MLL-AF9 driven AML.

MN1-driven leukemias that developed in ^{MN1}HSC-T and ^{MN1}CMP-T injected animals (termed ^{MN1}HSC-L and ^{MN1}CMP-L) were similar in clinical presentation with minor distinguishing features (FIG. 12A and FIG. 12B). We observed a trend towards lower expression of myeloid differentiation markers (CD11b, G1) and higher expression of cKit in ^{MN1}HSC-L, however, this was not statistically significant (FIG. 12E). We performed secondary transplants of ^{MN1}HSC-L and ^{MN1}CMP-L. In secondary recipients, there were no differences in penetrance or latency (FIG. 5C). A trend towards a more immature flow profile was also observed in HSC-derived secondary leukemias, however, the differences were small (FIG. 12C-E).

### Example 7

### ^{MN1}HSC-T Growth and HoxA9/Meis1 Expression are Independent of Dot1l In Vitro

Next, the question of whether ^{MN1}HSC-T are dependent on Dot1l in vitro, similar to what we observed for ^{MN1}CMP-T, was studied. Surprisingly, ^{MN1}HSC-T grew very well in vitro in complete absence of functional Dot1l (FIG. 6A). There was no increase in differentiation or apoptosis, no decrease in cell cycle in Dot1l^{-/- MN1}HSC-T compared to Dot1l wild type (FIG. 13A-C), and Dot1l^{-/- MN1}HSC-T were capable of forming blast like colonies in methylcellulose (FIG. 6B, left panel). Serial genotyping PCR of sorted bulk cultures confirmed persistence of both deleted alleles specifically in ^{MN1}HSC-T. In the same assay, ^{MN1}CMP-T derived leukemias show progressive outgrowth of non-deleted clones, confirming selective pressure against Dot1l^{-/- MN1}CMP-T but not Dot1l^{-/- MN1}HSC-T in vitro (FIG. 13D). In contrast to MN1-transformed LT-HSCs, MLL-AF9 transformed LT-HSCs (which readily caused leukemia in mice) require functional Dot1l: Dot1l^{-/- MLL-AF9}HSC-T formed less cellular and more dispersed colonies than Dot1l^{f/f MLL-AF9}HSC-T, similar to what was preciously observed with lineage depleted or LSK derived MLL-AF9 transformed cells (FIG. 6B, right panels, and Bernt et al, 2011). MLL-AF9 transformed LT-HSCs also require Dot1l in vivo (FIG. 13E). Genotyping of bulk cultures beyond the second replating confirmed selective pressure against the deleted allele in ^{MLL-AF9}HSC-T but not ^{MN1}HSC-T (FIG. 6C). Next the transcriptional consequences of loss of Dot1l in ^{MN1}HSC-T and ^{MN1}CMP-T were studied. MN1 transformed HSCs had previously been reported to express lower levels of HoxA9 than MN1 transformed CMPs (Heuser et al., 2011), and the instant data confirm this result (FIG. 6E). Consistent with the lack of phenotypic changes, no statistically significant changes in HoxA9 and Meisl expression in ^{MN1}HSC-T were found.

### Example 8

### MN1 Leukemias Derived from HSC-Enriched Populations Require Dot1l In Vivo

The finding that MN1 transformed sorted HSC-enriched populations grow independently of Dot1l in vitro and may be able to cause in vivo leukemia in recipients animals could have critical implications for patients, in whom the cell of origin is not known. Next the question of whether the leukemias originating from HSC-enriched populations recapitulated the phenotypic and transcriptional features we observed in vitro, with Dot1l-independent growth, a lack of selective pressure against the Dot1l-deleted allele, and low, Dot1l-independent HoxA9 expression was studied. In contrast to in vitro observations, Dot1l appeared to be required in vivo: animals injected with Cre-transduced ^{MN1}HSC-T (Dot1l^{-/-}) had lower leukemic burden than animals injected with control vector transduced ^{MN1}HSC-T (Dot1l^{f/f}). Cre-transduced ^{MN1}HSC-T (Dot1^{-/-}) caused leukemia with a trend towards increased latency and decreased penetrance (FIG. 6G and FIG. 13F). Most strikingly, the leukemias that did develop in the Dot1l^{-/- MN1}BSC-T group had failed to rearrange at least one Dot1l allele. Despite robust in vitro growth of Dot1l^{-/- MN1}HSC-T, not even a single Dot1l^{-/- MN1}HSC derived leukemia was observed. Next, Dot1l^{f/f} leukemias established from MN1 transduced HSC-enriched populations (Dot1l^{f/f MN1}HSC-L) were characterized. In contrast to results obtained with in vitro transformed ^{MN1}HSC-Ts, HoxA9 expression in in vivo ^{MN1}HSC-Ls was Dotll-dependent (FIG. 6H). As in primary recipients, no Dot1l^{-/- MN1}HSC-L in secondary recipients was observed (FIG. 6I and FIG. 13G).

### Example 9

### High HOXA9 Expression is Observed in a Subgroup of AML Patient Samples with high MN1 Expression

Results from the Dot1l conditional mouse model suggest that MN1-driven leukemias are dependent on high levels of endogenous HoxA9 expression, which in turn is dependent on functional Dot1l. This raises the possibility that targeting the MN1-cooperating program via inhibition of DOT1L could have therapeutic efficacy in MN1^{high} AML. However, high MN1 expression in clinical AML is observed over a broad range of phenotypic, cytogenetic and molecular subgroups, a heterogeneity that is not well captured in the retroviral MN1-overexpression mouse model. In order to investigate a potential role of DOT1L in clinical MN1^{high} AML, the question of whether HOXA9 and MEIS1 are co-expressed with MN1 in a substantial number of primary AML patient samples was studied. qPCR analysis of MN1 and HOXA9 and MEIS1 was performed on diagnostic RNA of 25 AML patient samples. MN1 is shown dichotomized at the median, the most commonly used cut-off to correlate MN1 with cytogenetics and outcome (FIG. 7A). HOXA9 expression was observed in 6 out of 11 AML samples with high MN1 expression, three of which were in a range comparable to samples with MLL-rearrangements (AML 38, 19 and 29; FIG. 7A, right axis). Elevated MEIS1 expression was observed in all HOXA9 expressing samples (FIG. 14B). Moderately high MEIS1 expression was also observed in several HOXA9 negative samples, including those with inv(16) (FIG. 14A- C). Correlation with cytogenetics revealed that two of the three samples with high MN1/HOXA9 expression had a complex karyotype with loss of 5q and/or 7q sequences (5q-/7q-, AML 38 and 19). On the other hand, 5 out of 11 AML samples with high MN1 expression had no detectable HOXA9/MEIS1 expression. The highest MN1 expression level in this group was observed in a sample with inv(16) (AML 2), which has previously been shown to be universally associated with MN1-overexpression (Carella et al., 2007; Haferlach et al., 2012). Analysis of a well annotated publicly available data set confirmed these results in a larger cohort of patients (Wouters et al., 2009b). The highest MN1 expression was found to be associated with two distinct cytogenetic subgroups, inv(16), and 5q-/7q-. As in the smaller cohort, HOXA9 was overexpressed in 5q-/7q-, but not inv(16) AML (FIG. 7B). AML with complex karyotype and 5q-/7q- often arises from myelodysplastic syndrome and is associated with poor outcome.

### Example 10

### 2 MN1^{high}/HOXA9^{high} Human AML Samples are Sensitive to DOT1L Inhibition

Next, the question of whether MN1^{high}/HOXA9^{high}5q-/7q- AML samples are sensitive to pharmacologic inhibition of DOT1L was studied. Viably frozen cells from AML 38 were maintained in culture on a feeder layer as recently described by Klco et al. (Klco et al., 2013) and exposed to the DOT1L inhibitor EPZ4777 in vitro (Daigle et al., 2011). EPZ4777 induced a dose dependent decrease in cell growth and in the fraction of cycling cells, as well as an increase in apoptosis (FIG. 7E). The observed effect was in a range comparable to AML 12 (MLL-rearranged, FIG. 7D), while AML 24 (AML/ETO, FIG. 7C) was unaffected. No viably frozen cells were available from the other two MN1^{high}/HOXA9^{high} samples in the initial cohort. Additional samples were screened by qPCR and identified a second MN1^{high}/HOXA9^{high} AML for in vitro exposure to EPZ4777 (AML 123009, FIG. 7F). Again, a dose dependent decrease in cell growth and S-phase, as well as an increase in apoptosis was observed. A dose-dependent upregulation of CD14, as well as a decrease in the nucleus:cytoplasma (N:C) ratio and increased vacuolization on cytospin consistent with differentiation was also observed (FIG. 7F). No diagnostic information was available for this sample. A standard cytogenetic analysis, which revealed a complex karyotype with 5q-, was performed.

### Example 11

### MN1 Expression and DOT1L Dependence

The response of non-MLL-rearranged cell lines with different HOXA9 and MN1 expression levels to DOT1L inhibition was assessed. Four cell lines with HOXA9 expression at the same or higher level as MLL-rearranged control cell lines were identified (FIG. 15A). Three of these cell lines, Loucy, KG1, and KG1a failed to show any phenotypic response to DOT1L inhibition. Complete inhibition of H3K79 methylation was verified by Western Blotting (all cell lines) and ChIP-Seq (Loucy only). Loucy, KG1 and KGla display MN1 expression levels that are similar to slightly elevated compared to MLL-rearranged cell lines. In contrast, MN1 expression in the Mutz3 cell line was 3000-fold higher than in MLL-rearranged control cell lines. Mutz3 responded to DOT1L inhibition (FIG. 15B). KG1 and KG1a cells were co-treated with cyclosporine A to inhibit MDR1, and complete inhibition of H3K79 methylation was confirmed by Western Blotting and ChIP-Seq.

A second subtype of AML that has been shown to display high MN1 expression levels are leukemias with inv(16). These leukemias do not typically express high levels of HOXA cluster genes. The inv(16) cell line Mel responds to DOT1L inhibition. Two inv(16) patient samples were characterized. Both samples also responded to DOT1L inhibition (example of sensitive sample shown in FIG. 15C).

A genetic loss of function model for Mll1 was used to investigate DOT1L dependence in MN1 driven leukemia. Deletion of Mll1 in MN1-transformed common myeloid progenitors (CMPs) resulted in decreased growth, and eventually exhaustion of serial replating. Mice injected with mostly (around 90%) Mll^{-/-} MN1-transduced CMPs failed to develop Mll^{-/-} leukemia. Although not resulting in a statistically significant survival advantage, all leukemias that developed in MLL^{-/-} injected mice originated from contaminating Mll^{f/f} cells, supporting strong selective pressure against the deleted allele in vivo (FIG. 16B and FIG. 16C). These data provide additional support that inhibiting DOT1L modulates a normal HSPC program.

In some embodiments, aberrant, leukemogenic expression of an MLL (sub)program could be achieved either through an MLL-rearrangement (fusion or PTD), or overexpression of the co-regulator MN1, as illustrated in the model shown in FIG. 16A. In this non-limiting model, MN1 is a transcriptional co-activator. Locus specific binding of MN1 is mediated by indirect interaction with a sequence specific transcription factor via p300/CBP (CREB binding protein). In this non-limiting model, the transcription factor in MN1-driven HOXA9high AML is wild type MLL1. MLL1 has been shown to interact with CBP in a developmental context. MN1 overexpression impairs the developmentally appropriate shut down of MLL1 target genes at the CMP to GMP transition in a similar fashion as MLL-fusions. This would suggest that in some embodiments MLL1 may be required for MN1 driven AML.

### REFERENCES

1. Bernt, K. M., Zhu, N., Sinha, A. U., Vempati, S., Faber, J., Krivtsov, A. V., Feng, Z., Punt, N., Daigle, A., Bullinger, L., et al. (2011). MLL-rearranged leukemia is dependent on aberrant H3K79 methylation by DOT1L. Cancer cell 20, 66-78.
2. Boiers, C., Carrelha, J., Lutteropp, M., Luc, S., Green, J. C., Azzoni, E., Woll, P. S., Mead, A. J., Hultquist, A., Swiers, G., et al. (2013). Lymphomyeloid contribution of an immune-restricted progenitor emerging prior to definitive hematopoietic stem cells. Cell stem cell 13, 535-548.
3. Buijs, A., Sherr, S., van Baal, S., van Bezouw, S., van der Plas, D., Geurts van Kessel, A., Riegman, P., Lekanne Deprez, R., Zwarthoff, E., Hagemeijer, A., and et al. (1995). Translocation (12;22) (p13;q11) in myeloproliferative disorders results in fusion of the ETS-like TEL gene on 12p13 to the MN1 gene on 22q11. Oncogene 10, 1511-1519.
4. Carella, C., Bonten, J., Sirma, S., Kranenburg, T. A., Terranova, S., Klein-Geltink, R., Shurtleff, S., Downing, J. R., Zwarthoff, E. C., Liu, P. P., and Grosveld, G. C. (2007). MN1 overexpression is an important step in the development of inv(16) AML. Leukemia 21, 1679-1690.
5. Cozzio, A., Passegue, E., Ayton, P. M., Karsunky, H., Cleary, M. L., and Weissman, I. L. (2003). Similar MLL-associated leukemias arising from self-renewing stem cells and short-lived myeloid progenitors. Genes & development 17, 3029-3035.
6. Daigle, S. R., Olhava, E. J., Therkelsen, C. A., Basavapathruni, A., Jin, L., Boriack-Sjodin, P. A., Allain, C. J., Klaus, C. R., Raimondi, A., Scott, M. P., et al. (2013). Potent inhibition of DOT1L as treatment of MLL-fusion leukemia. Blood 122, 1017-1025.
7. Daigle, S. R., Olhava, E. J., Therkelsen, C. A., Majer, C. R., Sneeringer, C. J., Song, J., Johnston, L. D., Scott, M. P., Smith, J. J., Xiao, Y., et al. (2011). Selective killing of mixed lineage leukemia cells by a potent small-molecule DOT1L inhibitor. Cancer cell 20, 53-65.
8. Essers, M. A., Offner, S., Blanco-Bose, W. E., Waibler, Z., Kalinke, U., Duchosal, M. A., and Trumpp, A. (2009). IFNalpha activates dormant haematopoietic stem cells in vivo. Nature 458, 904-908.
9. Factor, D. C., Corradin, O., Zentner, G. E., Saiakhova, A., Song, L., Chenoweth, J. G., McKay, R. D., Crawford, G. E., Scacheri, P. C., and Tesar, P. J. (2014). Epigenomic Comparison Reveals Activation of “Seed” Enhancers during Transition from Naive to Primed Pluripotency. Cell stem cell 14, 854-863.
10. Grosveld, G. C. (2007). MN1, a novel player in human AML. Blood cells, molecules & diseases 39, 336-339.
11. Guo, G., Luc, S., Marco, E., Lin, T. W., Peng, C., Kerenyi, M. A., Beyaz, S., Kim, W., Xu, J., Das, P. P., et al. (2013). Mapping cellular hierarchy by single-cell analysis of the cell surface repertoire. Cell stem cell 13, 492-505.
12. Haferlach, C., Kern, W., Schindela, S., Kohlmann, A., Alpermann, T., Schnittger, S., and Haferlach, T. (2012). Gene expression of BAALC, CDKN1B, ERG, and MN1 adds independent prognostic information to cytogenetics and molecular mutations in adult acute myeloid leukemia. Genes, chromosomes & cancer 51, 257-265.
13. Heuser, M., Argiropoulos, B., Kuchenbauer, F., Yung, E., Piper, J., Fung, S., Schlenk, R. F., Dohner, K., Hinrichsen, T., Rudolph, C., et al. (2007). MN1 overexpression induces acute myeloid leukemia in mice and predicts ATRA resistance in patients with AML. Blood 110, 1639-1647.
14. Heuser, M., Beutel, G., Krauter, J., Dohner, K., von Neuhoff, N., Schlegelberger, B., and Ganser, A. (2006). High meningioma 1 (MN1) expression as a predictor for poor outcome in acute myeloid leukemia with normal cytogenetics. Blood 108, 3898-3905.
15. Heuser, M., Yun, H., Berg, T., Yung, E., Argiropoulos, B., Kuchenbauer, F., Park, G., Hamwi, I., Palmqvist, L., Lai, C. K., et al. (2011). Cell of origin in AML: susceptibility to MN1-induced transformation is regulated by the MEIS1/AbdB-like HOX protein complex. Cancer cell 20, 39-52.
16. Jo, S. Y., Granowicz, E. M., Maillard, I., Thomas, D., and Hess, J. L. (2011). Requirement for Dotll in murine postnatal hematopoiesis and leukemogenesis by MLL translocation. Blood 117, 4759-4768.
17. Klco, J. M., Spencer, D. H., Lamprecht, T. L., Sarkaria, S. M., Wylie, T., Magrini, V., Hundal, J., Walker, J., Varghese, N., Erdmann-Gilmore, P., et al. (2013). Genomic impact of transient low-dose decitabine treatment on primary AML cells. Blood 121, 1633-1643.
18. Krivtsov, A. V., Figueroa, M. E., Sinha, A. U., Stubbs, M. C., Feng, Z., Valk, P. J., Delwel, R., Dohner, K., Bullinger, L., Kung, A. L., et al. (2013). Cell of origin determines clinically relevant subtypes of MLL-rearranged AML. Leukemia 27, 852-860.
19. Krivtsov, A. V., Twomey, D., Feng, Z., Stubbs, M. C., Wang, Y., Faber, J., Levine, J. E., Wang, J., Hahn, W. C., Gilliland, D. G., et al. (2006). Transformation from committed progenitor to leukaemia stem cell initiated by MLL-AF9. Nature 442, 818-822.
20. Langer, C., Marcucci, G., Holland, K. B., Radmacher, M. D., Maharry, K., Paschka, P., Whitman, S. P., Mrozek, K., Baldus, C. D., Vij, R., et al. (2009). Prognostic importance of MN1 transcript levels, and biologic insights from MN1-associated gene and microRNA expression signatures in cytogenetically normal acute myeloid leukemia: a cancer and leukemia group B study. Journal of clinical oncology : official journal of the American Society of Clinical Oncology 27, 3198-3204.
21. Lekanne Deprez, R. H., Riegman, P. H., Groen, N. A., Warringa, U. L., van Biezen, N. A., Molijn, A. C., Bootsma, D., de Jong, P. J., Menon, A. G., Kley, N. A., and et al. (1995). Cloning and characterization of MN1, a gene from chromosome 22q11, which is disrupted by a balanced translocation in a meningioma. Oncogene 10, 1521-1528.
22. Metzeler, K. H., Dufour, A., Benthaus, T., Hummel, M., Sauerland, M. C., Heinecke, A., Berdel, W. E., Buchner, T., Wormann, B., Mansmann, U., et al. (2009). ERG expression is an independent prognostic factor and allows refined risk stratification in cytogenetically normal acute myeloid leukemia: a comprehensive analysis of ERG, MN1, and BAALC transcript levels using oligonucleotide microarrays. Journal of clinical oncology: official journal of the American Society of Clinical Oncology 27, 5031-5038.
23. Nguyen, A. T., He, J., Taranova, 0., and Zhang, Y. (2011). Essential role of DOT1L in maintaining normal adult hematopoiesis. Cell research 21, 1370-1373.
24. van Wely, K. H., Meester-Smoor, M. A., Janssen, M. J., Aarnoudse, A. J., Grosveld, G. C., and Zwarthoff, E. C. (2007). The MN1-TEL myeloid leukemia-associated fusion protein has a dominant-negative effect on RAR-RXR-mediated transcription. Oncogene 26, 5733-5740.
25. Wouters, B. J., Lowenberg, B., and Delwel, R. (2009a). A decade of genome-wide gene expression profiling in acute myeloid leukemia: flashback and prospects. Blood 113, 291-298.
26. Wouters, B. J., Lowenberg, B., Erpelinck-Verschueren, C. A., van Putten, W. L., Valk, P. J., and Delwel, R. (2009b). Double CEBPA mutations, but not single CEBPA mutations, define a subgroup of acute myeloid leukemia with a distinctive gene expression profile that is uniquely associated with a favorable outcome. Blood 113, 3088-3091.
27. Xiang, L., Li, M., Liu, Y., Cen, J., Chen, Z., Zhen, X., Xie, X., Cao, X., and Gu, W. (2013). The clinical characteristics and prognostic significance of MN1 gene and MN1-associated microRNA expression in adult patients with de novo acute myeloid leukemia. Annals of hematology 92, 1063-1069.

What is claimed is:

1. A method of treating acute myeloid leukemia (AML), the method comprising administering a composition comprising a DOT1L inhibitor to a subject having one or more deletions in the 5q and/or 7q chromosomal regions.

2. The method of claim 1, wherein the subject has one or more deletions in the 5q and 7q chromosomal regions.

3. The method of claim 1 or 2, wherein the subject is identified as having one or more deletions in the 5q and/or 7q chromosomal regions.

4. The method of claim 3, wherein the one or more deletions in the 5q and/or 7q chromosomal regions are identified in a sample obtained from the subject.

5. A method of treating a subject having AML, the method comprising:
a. obtaining a biological sample from the subject;
b. detecting the presence of one or more deletions in the 5q and/or 7q chromosomal regions in the biological sample; and,
c. administering to the subject a composition comprising a DOT1L inhibitor.

6. The method of claim 5, wherein the subject has one or more deletions in the 5q and 7q chromosomal regions.

7. A method of treating AML, the method comprising administering a composition comprising a DOT1L inhibitor to a subject having MN1 and HOXA9 overexpression.

8. The method of claim 7, wherein the subject is identified as having MN1 and HOXA9 overexpression.

9. The method of claim 8, wherein the MN1 and HOXA9 overexpression are detected in a sample obtained from the subject.

10. A method of treating a subject having AML, the method comprising:
a. obtaining a biological sample from the subject;
b. detecting overexpression of MN1 and HOXA9 in the biological sample; and, c. administering to the subject a composition comprising a DOT1L inhibitor.

11. The method of any one of claims 7 to 10, wherein the subject has deletions in the 5q and/or 7q chromosomal regions.

12. The method of any one of claims 1 to 11, wherein the DOT1L inhibitor is a compound of formula: 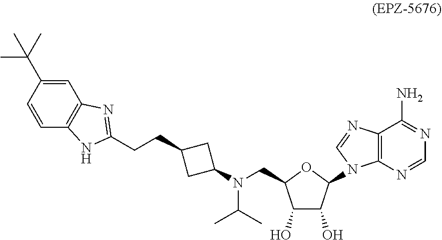 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]
or a pharmaceutically acceptable salt thereof.

13. The method of any one of claims 1 to 11, wherein the DOT1L inhibitor is a compound of formula: 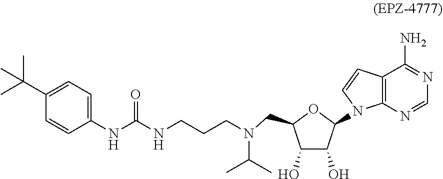 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]
or a pharmaceutically acceptable salt thereof.

14. The method of claim 5 or 10, wherein the biological sample is selected from the group consisting of bone marrow, peripheral blood cells, blood, cerebrospinal fluid, skin lesions, chloroma biopsy, plasma, serum, urine, saliva and a cell.

15. The method of claim 5, wherein the presence of one or more deletions in the 5q and/or 7q chromosomal regions in the biological sample is detected by a cytogenetic method.

16. The method of claim 15, wherein the cytogenetic method is karyotyping.

17. The method of claim 15, wherein the cytogenetic method is fluorescent in situ hybridization (FISH) or comparative genomic hybridization (CGH).

18. The method of claim 5, wherein the presence of one or more deletions in the 5q and/or 7q chromosomal regions in the biological sample is detected by polymerase chain reaction (PCR).

19. The method of claim 9 or 10, wherein MN1 and HOXA9 overexpression in the biological sample is detected in a gene expression assay.

20. The method of claim 19, wherein the gene expression assay comprises a hybridization assay, a nucleic acid amplification, a quantitative PCR (qPCR) analysis, an RNA-Seq, or a Northern blot analysis.

21. The method of claim 19, wherein the gene expression assay comprises a microarray analysis.

22. The method of claim 9 or 10, wherein MN1 and HOXA9 overexpression in the biological sample is detected in a protein expression assay.

23. The method of claim 22, wherein the protein expression assay comprises a Western Blot, ELISA, or other antibody-based protein expression assay.

24. A method of identifying a subject having AML that is responsive to treatment with a DOT1L inhibitor, the method comprising:
a. obtaining a biological sample from the subject;
b. detecting the presence of one or more deletions in the 5q and/or 7q chromosomal regions in the biological sample; and,
c. identifying the subject as responsive to treatment with a DOT1L inhibitor if one or more deletions in the 5q and/or 7q chromosomal regions are detected in the biological sample.

25. A method of identifying a subject having AML that is responsive to treatment with a DOT1L inhibitor, the method comprising:
a. obtaining a biological sample from the subject;
b. detecting the overexpression of MN1 and HOXA9 in the biological sample; and,
c. identifying the subject as responsive to treatment with a DOT1L inhibitor if overexpression of MN1 and HOXA9 is detected in the biological sample.
